(19) United States
(12) Patent Application Publication
(10) Pub. No.: US 2026/0216151 A1
(43) Pub. Date: Jul. 30, 2026

(54) GPR65 MODULATORS

(71) Applicant: Pathios Therapeutics Limited, Abingdon, Oxford Oxfordshire (GB)

(72) Inventors: Alan Naylor, Oxfordshire (GB); David Miller, Oxfordshire (GB); Gavin Milne, Oxfordshire (GB)

(73) Assignee: Pathios Therapeutics Limited, Abingdon, Oxford Oxfordshire, GB (GB)

(21) Appl. No.: 19/152,577

(22) PCT Filed: Feb. 12, 2024
(86) PCT No.: PCT/GB2024/050375

§ 371 Date: Jul. 31, 2025

(30) Foreign Application Priority Data
Feb. 13, 2023 (GB) 2302032.4

(51) Int. Cl. A61K 31/438; A61K 31/5025; A61P 35/00; C07D 471/04; C07D 519/00

(52) U.S. Cl. CPC A61K 31/438; A61K 31/5025; A61P 35/00; C07D 471/04; C07D 519/00

(57) ABSTRACT

The present invention relates to a compound of formula (I), or a pharmaceutically acceptable salt or solvate thereof, wherein: ring A is a 5- or 6-membered monocyclic aromatic or heteroaromatic ring, or a 9- or 10-membered bicyclic aromatic or heteroaromatic ring, each of which is optionally substituted with one or more substituents independently selected from halo, CN, alkoxy, NR₁₁R_{11′}, OH, SO₂-alkyl, CO₂-alkyl, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl; Y and Z are each independently CR₁₀R_{10′}, wherein R₁₀ and R_{10′} are each independently selected from H, F, alkyl, and haloalkyl; R₁, R₄, and R₅ are each independently selected from H, alkyl, alkoxy, O-aralkyl halo and haloalkyl; at least one of R₂ and R₃ is a monocyclic or fused bicyclic aryl or a monocyclic or fused bicyclic heteroaryl group, each of which is optionally substituted by one or more substituents selected from alkyl, alkoxy, haloalkyl, haloalkoxy, halo, aryl, heteroaryl, CONR₁₄R_{14′}, (CH₂)mNR₁₁R_{11′}, SO₂-alkyl, CN, hydroxyalkyl, (CH₂)m-heterocycloalkyl, (CH₂)ₘ-cycloalkyl, CO₂R₁₅, alkoxy-alkyl and O-cycloalkyl; the other of R₂ and R₃ is selected from halo, CN, and haloalkyl; each R₁₁ and each R_{11′} is independently selected from H, alkyl, haloalkyl, COR₁₂, and SO₂R₁₃, wherein R₁₂ and R₁₃ are both alkyl; R₁₄, R_{14′} and Rw are each independently selected from H, alkyl and alkoxyalkyl; and each m is independently 0 to 3. Further aspects of the invention relate to such compounds for use in the field of immuno-oncology, immunology, and related applications.

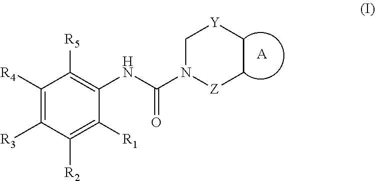

The present invention relates to compounds that are capable of modulating GPR65. The compounds have potential therapeutic applications in the treatment of a variety of disorders including proliferative and immune disorders.

### BACKGROUND TO THE INVENTION

GPR65 is a Gs-coupled G protein-coupled receptor (GPCR) that is primarily expressed in immune cells and is activated by acidic extracellular pH to cause increases in cytoplasmic cyclic adenosine monophosphate (cAMP) (Wang, 2004). It has long been known that tumours typically undergo a switch in cellular metabolism from oxidative phosphorylation to aerobic glycolysis, which in turn results in an acidic extracellular microenvironment (Damaghi, 2013). Recently, it has been shown that this acidic microenvironment causes GPR65 activation in tumour-associated macrophages, resulting in an increase in cytoplasmic cAMP leading to transcription of the inducible cAMP early repressor (ICER). This, in turn, suppresses the secretion of tumour necrosis factor alpha (TNFα) to bias the macrophages toward an anti-inflammatory, tumour-permissive phenotype (Bohn, 2018). This GPR65-dependent pathway therefore appears to represent a mechanism by which tumours exploit their acidic microenvironment to evade detection by the immune system.

Autoimmune diseases are also often associated with an acidic local microenvironment (for instance, an inflamed joint). Recent studies also suggest that GPR65 acts through ICER in CD4+ T cells, to suppress IL-2 and hence bias cells toward an inflammatory Th17 phenotype, which is associated with increased pathogenicity in the context of autoimmune disease (Korn, 2009). Supporting this is the recent finding that ICER is required for Th17 differentiation (Yoshida, 2016) as well as that agonism of GPR65 leads to an increase in Th17 differentiation (Hernandez, 2018). Indeed, mutations in the GPR65 locus are associated with several autoimmune diseases, such as multiple sclerosis, ankylosing spondylitis, inflammatory bowel disease, and Crohn's disease (Gaublomme, 2015). One recent study found that mice with CD4+ T cells lacking GPR65 were protected from developing the disease autoimmune encephalomyelitis (EAE) (Gaublomme, 2015).

Thus, GPR65 appears to act through ICER to promote an anti-inflammatory and tumour-permissive phenotype in tumour associated macrophages and an inflammatory Th17 phenotype in CD4+ T cells that is associated with autoimmune disease. GPR65 signalling, therefore, represents an attractive pathway for therapeutic intervention for the treatment of both cancer and autoimmune diseases. There is therefore an ongoing need to develop new small molecule GPR65 modulators.

The present invention seeks to provide compounds that are capable of modulating GPR65. As made clear from the above discussion, such compounds have potential therapeutic applications in the treatment of a variety of disorders, including proliferative disorders and immune disorders as well as asthma and chronic obstructive pulmonary disease.

### STATEMENT OF INVENTION

A first aspect of the invention relates to a compound of formula (I), or a pharmaceutically acceptable salt or solvate thereof,

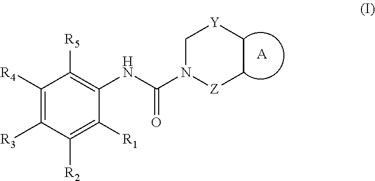

wherein:
ring A is a 5- or 6-membered monocyclic aromatic or heteroaromatic ring, or a 9- or 10-membered bicyclic aromatic or heteroaromatic ring, each of which is optionally substituted with one or more substituents independently selected from halo, CN, alkoxy, NR₁₁R₁₁′, OH, SO₂-alkyl, CO₂-alkyl, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R₁₁′, OH, alkyl, haloalkyl, and aralkyl;
Y and Z are each independently CR₁₀R₁₀′, wherein R₁₀ and R₁₀′ are each independently selected from H, F, alkyl, and haloalkyl;
R₁, R₄, and R₅ are each independently selected from H, alkyl, alkoxy, O-aralkyl, halo and haloalkyl;
at least one of R₂ and R₃ is a monocyclic or fused bicyclic aryl or a monocyclic or fused bicyclic heteroaryl group, each of which is optionally substituted by one or more substituents selected from alkyl, alkoxy, haloalkyl, haloalkoxy, halo, aryl, heteroaryl, CONR₁₄R₁₄′, (CH₂)ₘNR₁₁R₁₁′, SO₂-alkyl, CN, hydroxyalkyl, (CH₂)ₘ-heterocycloalkyl, (CH₂)ₘ-cycloalkyl, CO₂R₁₅, alkoxy-alkyl and O-cycloalkyl;
the other of R₂ and R₃ is selected from halo, CN, and haloalkyl;
each R₁₁ and each R₁₁′ is independently selected from H, alkyl, haloalkyl, COR₁₂, and SO₂R₁₃, wherein R₁₂ and R₁₃ are both alkyl;
R₁₄, R₁₄′ and R₁₅ are each independently selected from H, alkyl and alkoxyalkyl; and
each m is independently 0 to 3;
   with the proviso that the compound is other than:
N-(3-fluoro-4-(4-methyl-1H-imidazol-1-yl)phenyl)-6-methoxy-3,4-dihydroisoquinoline-2(1H)-carboxamide;
N-(3-fluoro-4-(4-methyl-1H-imidazol-1-yl)phenyl)-3,4-dihydroisoquinoline-2(1H)-carboxamide;
N-(3-fluoro-4-(4-methyl-1H-imidazol-1-yl)phenyl)-6,7-dimethoxy-3,4-dihydroisoquinoline-2(1H)-carboxamide.

Advantageously, the presently claimed compounds are capable of modulating GPR65, thereby rendering the compounds of therapeutic interest in the treatment of various disorders, for example, in the fields of oncology, immuno-oncology, and immunology.

Another aspect of the invention relates to a pharmaceutical composition comprising a compound as described above and a pharmaceutically acceptable diluent, excipient, or carrier.

Another aspect of the invention relates to a compound or a pharmaceutical composition as described above for use as a medicament.

Another aspect of the invention relates to a compound or a pharmaceutical composition as described above for use in treating or preventing a disorder selected from a proliferative disorder, an immune disorder, asthma, chronic obstructive pulmonary disease (COPD) and acute respiratory distress syndrome (ARDS).

Another aspect of the invention relates to a method of treating a disorder, comprising administering to a subject a compound or a pharmaceutical composition as described above.

### DETAILED DESCRIPTION

The present invention relates to compounds that are capable of modulating GPR65.

“Alkyl” is defined herein as a straight-chain or branched alkyl radical, preferably C₁₋₂₀ alkyl, more preferably C₁₋₁₂ alkyl, even more preferably C₁₋₁₀ alkyl or C₁₋₆ alkyl, for example, methyl, ethyl, propyl, isopropyl, butyl, isobutyl, tert-butyl, pentyl, hexyl. More preferably, the alkyl is a C₁₋₃ alkyl.

As used herein, the term “aryl” refers to a C₆₋₁₂ aromatic group, which may be benzocondensed, for example, phenyl or naphthyl. Preferably, the aryl group is phenyl.

“Halogen” is defined herein as chloro, fluoro, bromo or iodo.

“Haloalkyl” is defined herein as a straight-chain or branched alkyl radical as defined above, for example, methyl, ethyl, propyl, isopropyl, butyl, isobutyl, tert-butyl, pentyl, hexyl, that is substituted with one or more halogen atoms (that may be the same or different), such as fluorine, chlorine, bromine, and iodine. Preferably, the haloalkyl is a C₁₋₂₀ haloalkyl, more preferably a C₁₋₁₂ haloalkyl, even more preferably a C₁₋₁₀ haloalkyl or a C₁₋₆ haloalkyl, or a C₁₋₃ haloalkyl. Preferred examples are CF₃ and CHF₂, with CF₃ being particularly preferred.

“Alkoxy” is defined herein as an oxygen atom bonded to an alkyl group as defined above, for example methoxy, ethoxy, propoxy, isopropoxy, butoxy, isobutoxy, tert-butoxy, pentoxy and hexoxy. Preferably, the alkoxy is a C₁₋₂₀ alkoxy, more preferably a C₁₋₁₂ alkoxy, even more preferably C₁₋₁₀ alkoxy or a C₁₋₆ alkoxy, or a C₁₋₃ alkoxy. A preferred example is methoxy (—OCH₃).

“Haloalkyloxy” is defined herein as an alkoxy group as defined above substituted with one or more halogen atoms (that may be the same or different), such as fluorine, chlorine, bromine, and iodine.

“Cycloalkyl” is defined herein as a cyclic alkyl ring, preferably, C₃₋₇-cycloalkyl, more preferably C₃₋₆-cycloalkyl. Preferred examples include cyclopropyl, cyclobutyl, cyclopentyl, cyclohexyl or cycloheptyl, or a fused bicyclic ring system such as norbornane.

“Heteroaryl” is defined herein as a monocyclic or bicyclic C₂₋₁₂ aromatic ring comprising one or more heteroatoms (that may be the same or different), such as oxygen, nitrogen or sulphur. Examples of suitable heteroaryl groups include thienyl, furanyl, pyrrolyl, pyridinyl, oxazolyl, thiazolyl, imidazolyl, pyrazolyl, pyridazinyl, isoxazolyl, pyrimidinyl, pyrazinyl, triazinyl, isothiazolyl, oxadiazolyl, triazolyl, thiadiazolyl etc. and benzo derivatives thereof, such as benzofuranyl, benzothienyl, benzimidazolyl, indolyl, isoindolyl, indazolyl etc.; or pyridyl, pyrazinyl, pyrimidinyl, pyridazinyl, triazinyl etc. and benzo derivatives thereof, such as quinolinyl, isoquinolinyl, cinnolinyl, phthalazinyl, quinazolinyl, quinoxalinyl, naphthyridinyl etc.

“Heterocycloalkyl” refers to a cyclic aliphatic group containing one or more heteroatoms selected from nitrogen, oxygen and sulphur, which is optionally interrupted by one or more —(CO)— groups in the ring and/or which optionally contains one or more double bonds in the ring. Preferably, the heterocycloalkyl group is monocyclic or bicyclic. Preferably, the heterocycloalkyl group is saturated. Preferably, the heterocycloalkyl group is a C₃₋₇-heterocycloalkyl, more preferably a C₃₋₆-heterocycloalkyl. Alternatively, the heterocycloalkyl group is a C₄₋₇-heterocycloalkyl, more preferably a C₄₋₆-heterocycloalkyl. Preferred heterocycloalkyl groups include, but are not limited to, piperazinyl, piperidinyl, morpholinyl, thiomorpholinyl, pyrrolidinyl, tetrahydrofuranyl and tetrahydropyranyl.

“Aralkyl’ is defined herein as an alkyl group as defined above substituted by one or more aryl groups as defined above.

### Compounds According to the Invention

One aspect of the invention relates to compounds of formula (I′):

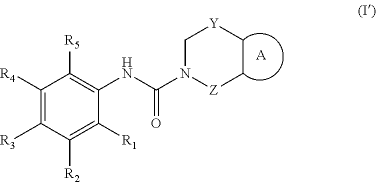

wherein:
ring A is a 5- or 6-membered monocyclic aromatic or heteroaromatic ring, or a 9- or 10-membered bicyclic aromatic or heteroaromatic ring, each of which is optionally substituted with one or more substituents independently selected from halo, CN, alkoxy, NR₁₁R₁₁′, OH, SO₂-alkyl, CO₂-alkyl, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R₁₁′, OH, alkyl, haloalkyl, and aralkyl;
Y and Z are each independently CR₁₀R₁₀′, wherein R₁₀ and R₁₀′ are each independently selected from H, F, alkyl, and haloalkyl;
R₁, R₄, and R₅ are each independently selected from H, halo and haloalkyl;
at least one of R₂ and R₃ is a monocyclic or fused bicyclic aryl or a monocyclic or fused bicyclic heteroaryl group, each of which is optionally substituted by one or more substituents selected from alkyl, alkoxy, haloalkyl, haloalkoxy, halo, aryl, heteroaryl, CONR₁₄R₁₄′, (CH₂)ₘNR₁₁R₁₁′, SO₂-alkyl, CN, hydroxyalkyl, (CH₂)ₘ-heterocycloalkyl, (CH₂)ₘ-cycloalkyl, CO₂R₁₅, alkoxy-alkyl and O-cycloalkyl;
the other of R₂ and R₃ is selected from H, halo, CN, and haloalkyl;
each R₁₁ and each R₁₁′ is independently selected from H, alkyl, haloalkyl, COR₁₂, and SO₂R₁₃, wherein R₁₂ and R₁₃ are both alkyl;
R₁₄, R₁₄′ and R₁₅ are each independently selected from H, alkyl and alkoxyalkyl; and
each m is independently 0 to 3.

One aspect of the invention relates to a compound of formula (I), or a pharmaceutically acceptable salt or solvate thereof,

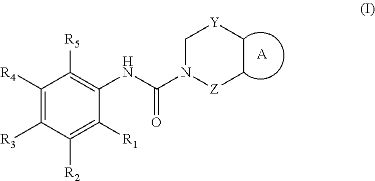

wherein:
ring A is a 5- or 6-membered monocyclic aromatic or heteroaromatic ring, or a 9- or 10-membered bicyclic aromatic or heteroaromatic ring, each of which is optionally substituted with one or more substituents independently selected from halo, CN, alkoxy, NR₁₁R₁₁′, OH, SO₂-alkyl, CO₂-alkyl, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R₁₁′, OH, alkyl, haloalkyl, and aralkyl;
Y and Z are each independently CR₁₀R₁₀′, wherein R₁₀ and R₁₀′ are each independently selected from H, F, alkyl, and haloalkyl;
R₁, R₄, and R₅ are each independently selected from H, alkyl, alkoxy, O-aralkyl, halo and haloalkyl;
at least one of R₂ and R₃ is a monocyclic or fused bicyclic aryl or a monocyclic or fused bicyclic heteroaryl group, each of which is optionally substituted by one or more substituents selected from alkyl, alkoxy, haloalkyl, haloalkoxy, halo, aryl, heteroaryl, CONR₁₄R₁₄′, (CH₂)ₘNR₁₁R₁₁′, SO₂-alkyl, CN, hydroxyalkyl, (CH₂)ₘ-heterocycloalkyl, (CH₂)ₘ-cycloalkyl, CO₂R₁₅, alkoxy-alkyl and O-cycloalkyl;
the other of R₂ and R₃ is selected from halo, CN, and haloalkyl;
each R₁₁ and each R₁₁′ is independently selected from H, alkyl, haloalkyl, COR₁₂, and SO₂R₁₃, wherein R₁₂ and R₁₃ are both alkyl;
R₁₄, R₁₄′ and R₁₅ are each independently selected from H, alkyl and alkoxyalkyl; and
each m is independently 0 to 3.

In compounds according to the invention, preferably alkyl is C₁-C₆ alkyl, haloalkyl is C₁-C₆ haloalkyl, and alkoxy is C₁-C₆ alkoxy. The preferred embodiments below apply equally to formula (I) and (I′).

In one preferred embodiment, R₁₀ and R₁₀′ are each independently selected from H, F, Me and CF₃. More preferably, R₁₀ and R₁₀′ are both H.

In one preferred embodiment, Y and Z are each independently selected from CH₂, CHMe, CHF, CF₂, C(CH₃)₂, C(CF₃)₂. In one preferred embodiment, one of Z and Y is CHMe and the other is CH₂. In one preferred embodiment, Y is CHMe and Z is CH₂. In one preferred embodiment, Z is CHMe and Y is CH₂.

In a more preferred embodiment, Z and Y are both CH₂, i.e. the compound is of the following formula:

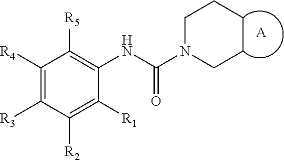

In one preferred embodiment, R₁, R₄, and R₅ are each independently selected from H, halo and haloalkyl.

In one preferred embodiment, R₁ is selected from H, haloalkyl and F, and is more preferably H. More preferably, R₁ is selected from H and F. In one particularly preferred embodiment, R₁ is H.

In one preferred embodiment, R₃ is a monocyclic or fused bicyclic aryl or a monocyclic or fused bicyclic heteroaryl group, each of which is optionally substituted by one or more substituents selected from alkyl, alkoxy, haloalkyl, haloalkoxy, halo, aryl, heteroaryl, CONR₁₄R₁₄′, (CH₂)ₘNR₁₁R₁₁′, SO₂-alkyl, CN, hydroxyalkyl, (CH₂)ₘ-heterocycloalkyl, (CH₂)ₘ-cycloalkyl, CO₂R₁₅, alkoxy-alkyl and O-cycloalkyl; and R₂ is selected from H, halo, CN, and haloalkyl, more preferably, halo, CN, and haloalkyl.

In one preferred embodiment, R₂ is selected from halo, CN and haloalkyl; and R₃ is an optionally substituted monocyclic aryl group; an optionally substituted monocyclic heteroaryl group; an optionally substituted fused bicyclic aryl group, or an optionally substituted fused bicyclic heteroaryl group.

In one preferred embodiment, R₃ is selected from halo, CN and haloalkyl; and R₂ is an optionally substituted monocyclic aryl group; an optionally substituted monocyclic heteroaryl group; an optionally substituted fused bicyclic aryl group, or an optionally substituted fused bicyclic heteroaryl group.

In one preferred embodiment, m is 0 or 1, preferably 0.

In one preferred embodiment, R₂ is H or halo, preferably halo, even more preferably, Cl.

In one preferred embodiment, R₃ is selected from phenyl, pyridinyl, pyrimidinyl, pyridazinyl, pyrazinyl, isoxazolyl, oxazolyl, thiazolyl, isothiazolyl, thiadiazoyl, triazinyl, thienyl, furanyl, pyrrolyl, imidazolyl, pyrazolyl, oxadiazolyl, triazolyl, benzofuranyl, benzothienyl, pyrrolopyridinyl, benzoxazinyl, benzimidazolyl, benzotriazolyl, benzoxadiazolyl, benzothiadiazolyl, benzothiazolyl, oxadiazolopyridinyl, indazolinyl, imidazopyridinyl, benzoisoxazolyl, benzoxazolyl, indolyl, isoindolyl, oxoisoindolinyl, oxoindolinyl, indazolyl, quinolinyl, isoquinolinyl, cinnolinyl, phthalazinyl, quinazolinyl, quinoxalinyl and naphthyridinyl, each of which is optionally substituted by one or more substituents selected from alkyl, alkoxy, haloalkyl, haloalkoxy and halo.

In one preferred embodiment, R₃ is selected from phenyl, pyridinyl, pyrimidinyl, pyridazinyl, pyrazinyl, isoxazolyl, oxazolyl, thiazolyl, isothiazolyl, thiadiazoyl, triazinyl, thienyl, furanyl, pyrrolyl, pyrazolyl, oxadiazolyl, triazolyl, benzofuranyl, benzothienyl, pyrrolopyridinyl, benzoxazinyl, benzimidazolyl, benzotriazolyl, benzoxadiazolyl, benzothiadiazolyl, benzothiazolyl, oxadiazolopyridinyl, indazolinyl, imidazopyridinyl, benzoisoxazolyl, benzoxazolyl, indolyl, isoindolyl, oxoisoindolinyl, oxoindolinyl, indazolyl, quinolinyl, isoquinolinyl, cinnolinyl, phthalazinyl, quinazolinyl, quinoxalinyl and naphthyridinyl, each of which is optionally substituted by one or more substituents selected from alkyl, alkoxy, haloalkyl, haloalkoxy and halo.

In one preferred embodiment, R₃ is selected from

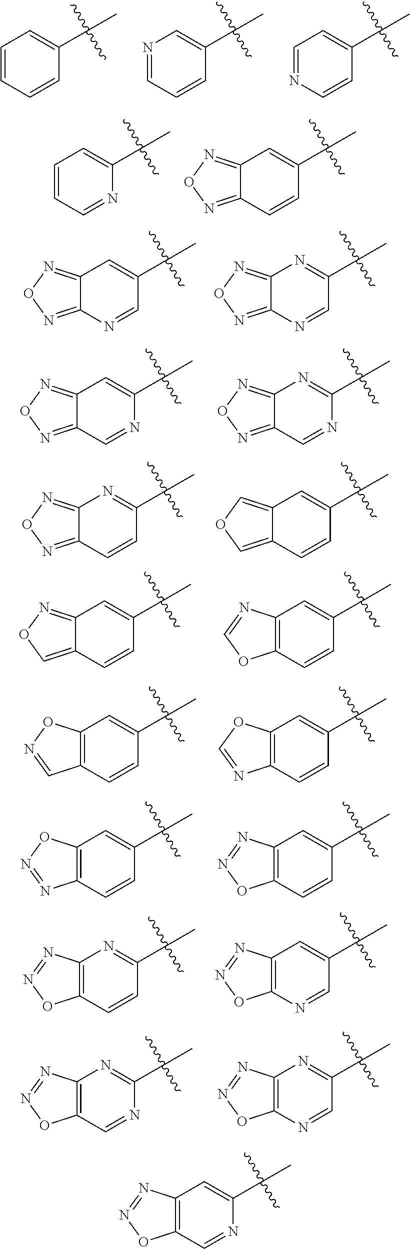

each of which is optionally substituted by one or more substituents selected from alkyl, halo, haloalkyl, alkoxy and haloalkoxy.

In one preferred embodiment, R₃ is selected from:

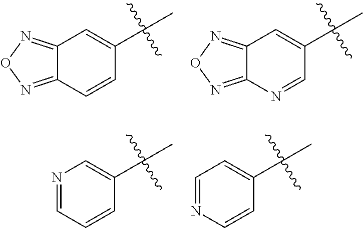 [SMILES: *c1ccc2nonc2c1 ; *c1cnc2nonc2c1 ; *c1cccnc1 ; *c1ccncc1]

each of which is optionally substituted by one or more substituents selected from alkyl, halo, haloalkyl, alkoxy and haloalkoxy. More preferably, the one or more substituents is selected from F, alkoxy and fluoroalkoxy.

In one preferred embodiment, R₃ is selected from:

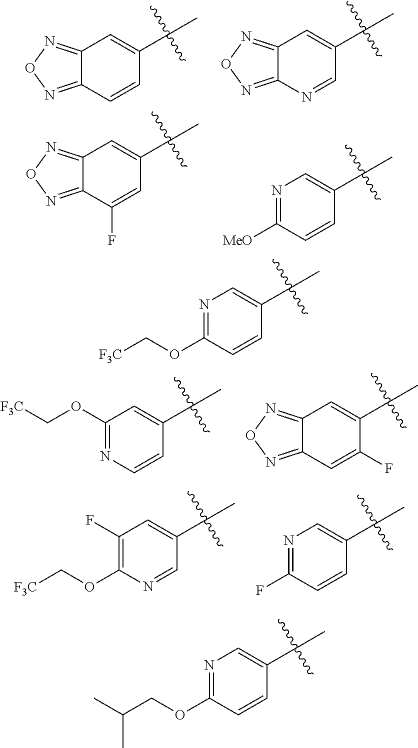

In one preferred embodiment, R₄ is H or Cl, and is more preferably H.

In one preferred embodiment, R₅ is H, alkyl, O-aralkyl or halo, preferably halo.

In one preferred embodiment, R₅ is H, Me, O-benzyl or F, preferably F.

In one preferred embodiment, R₅ is H or halo, preferably halo, even more preferably F.

In one preferred embodiment, R₁ and R₄ are both H, and R₂ and R₅ are both halo.

In one preferred embodiment, R₁ and R₄ are both H, R₂ is Cl and R₅ is F.

In the compounds described herein, ring A is a 5- or 6-membered monocyclic aromatic or heteroaromatic ring, or a 9- or 10-membered bicyclic aromatic or heteroaromatic ring, each of which is optionally substituted with one or more substituents independently selected from halo, CN, alkoxy, NR₁₁R₁₁′, OH, SO₂-alkyl, CO₂-alkyl, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R₁₁′, OH, alkyl, haloalkyl, and aralkyl.

In one embodiment, the compounds described herein contain an optionally substituted 5 or 6-membered monocyclic aromatic or heteroaromatic ring A fused to the nitrogen containing ring. The optional substituents are selected from halo, CN, alkoxy, NR₁₁R₁₁′, OH, SO₂-alkyl, CO₂-alkyl, alkyl, haloalkyl, aralkyl, aryl, and heteroaryl, wherein said aryl and heteroaryl substituent is in turn optionally substituted with one or more substituents each independently selected from halo, CN, alkoxy, NR₁₁R₁₁′, OH, alkyl, haloalkyl, and aralkyl.

In one preferred embodiment, ring A is an optionally substituted 6-membered aromatic group. In another preferred embodiment, ring A is an optionally substituted 5- or 6-membered heteroaromatic group.

In one preferred embodiment, ring A is optionally substituted with one or more substituents independently selected from halo, CN, NR₁₁R₁₁′, OH, SO₂-alkyl, CO₂-alkyl, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R₁₁′, OH, alkyl, haloalkyl, and aralkyl.

In one preferred embodiment, the monocyclic aromatic or heteroaromatic ring A is a group selected from benzene, pyridine, pyridone, pyridine N-oxide, pyridazine, pyrimidine, pyrimidone, pyrazine, triazine, pyrrole, furan, thiophene, pyrazole, isoxazole, imidazole, oxazole, oxadiazole and thiazole, each of which may be optionally substituted.

In one preferred embodiment, the monocyclic aromatic or heteroaromatic ring A is a group selected from benzene, pyridine, pyridone, pyridine N-oxide, pyrimidine, pyrimidone, pyridazine, pyrazine and isoxazole, each of which is optionally substituted.

In one embodiment, optional substituents on the A ring are selected from F, Cl, Br, I, CN, alkoxy, NR₁₁R₁₁′, OH, SO₂-alkyl, CO₂-alkyl, alkyl and haloalkyl.

In one preferred embodiment, ring A is optionally substituted by one or more substituents selected from halo, CN, C₁-C₆ alkoxy, NR₁₁R₁₁′, OH, C₁-C₆-alkyl, phenyl, SO₂—C₁-C₆ alkyl, CO₂—C₁-C₆ alkyl, thienyl, halo-substituted pyridinyl, and C₁-C₆-haloalkyl. More preferably, ring A is optionally substituted by one or more substituents selected from Me, Cl, F, CN, MeO, NH₂, OH, CO₂Me, SO₂Me, thienyl and fluoropyridinyl.

In some instances, ring A can exist in more than one tautomeric form. By way of illustration, where the heteroaromatic ring is substituted by an OH group, ring A can exist as two possible tautomers as shown below:

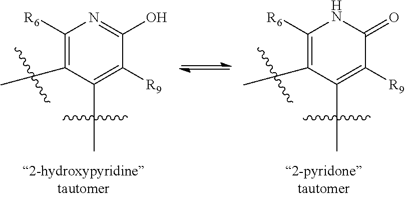

The 2-pyridone tautomer is believed to be the predominant solid state form. In solution, the energy difference between the two tautomeric forms is understood to be very small and is dependent on the polarity of the solvent. The skilled person would appreciate that other hydroxy substituted N-containing heteroaromatic groups (e.g. pyrimidine, other pyridine regioisomers) can be similarly represented in tautomeric form as shown above. The term “heteroaromatic” as used herein encompasses all tautomeric forms of the compounds.

In one preferred embodiment, ring A is a group selected from benzene, pyridine, pyridone, pyridine N-oxide, pyrimidine, pyrimidone, pyridazine, pyrazine, and isoxazole, each of which is optionally substituted with one or more substituents selected from F, Cl, Br, I, CN, C₁-C₆ alkoxy, NR₁₁R₁₁′, OH, C₁-C₆ alkyl, phenyl, SO₂-alkyl, CO₂-alkyl, thienyl, halo-substituted pyridinyl, and C₁-C₆ haloalkyl.

In one preferred embodiment, ring A is a group selected from benzene, pyridine, pyridone, pyridine N-oxide, pyrimidine, pyrimidone, pyridazine, pyrazine, and isoxazole, each of which is optionally substituted with one or more substituents selected from F, Cl, Br, I, CN, C₁-C₆ alkoxy, NR₁₁R₁₁′, OH, C₁-C₆ alkyl, SO₂-alkyl, CO₂-alkyl, I, and C₁-C₆ haloalkyl.

In one preferred embodiment, ring A is a benzene group which is optionally substituted with one or more substituents selected from F, Cl, Br, I, CN, C₁-C₆ alkoxy, NR₁₁R₁₁′, OH, C₁-C₆ alkyl, SO₂—C₁-C₆ alkyl, CO₂—C₁-C₆ alkyl, and C₁-C₆ haloalkyl.

In one preferred embodiment, ring A is a pyridine group which is optionally substituted with one or more substituents selected from F, Cl, Br, I, CN, C₁-C₆ alkoxy, NR₁₁R₁₁′, OH, C₁-C₆ alkyl, SO₂—C₁-C₆ alkyl, CO₂—C₁-C₆ alkyl, and C₁-C₆ haloalkyl.

In one preferred embodiment, ring A is a pyridone group which is optionally substituted with one or more substituents selected from F, Cl, Br, I, CN, C₁-C₆ alkoxy, NR₁₁R_{11′}, OH, C₁-C₆ alkyl, SO₂—C₁-C₆ alkyl, CO₂-C₁-C₆ alkyl, and C₁-C₆ haloalkyl.

In one preferred embodiment, ring A is an optionally substituted 10-membered bicyclic aromatic ring.

In one preferred embodiment, ring A is a 9- or 10-membered bicyclic heteroaromatic ring containing 1 to 4 nitrogen atoms, more preferably 1 to 3 nitrogen atoms. Preferably, the 9- or 10-membered bicyclic heteroaromatic ring containing 1 to 4 nitrogen atoms is selected from a triazolopyridine and an imidazopyridine, each of which is optionally substituted. More preferably, the 9- or 10-membered bicyclic heteroaromatic ring containing 1 to 4 nitrogen atoms is selected from [1,2,4]triazolo[4,3-a]pyridine, [1,2,4]triazolo[1,5-a]pyridine, imidazo[1,5-a]pyridine and imidazo[1,2-a]pyridine, each of which is optionally substituted.

Preferably, the 9- or 10-membered bicyclic heteroaromatic ring is optionally substituted by one or more substituents selected from halo, CN, C₁-C₆ alkoxy, NR₁₁R₁₁′, OH, C₁-C₆ alkyl, phenyl, SO₂—C₁-C₆ alkyl, CO₂—C₁-C₆ alkyl, thienyl, halo-substituted pyridinyl, and C₁-C₆ haloalkyl. More preferably, the 9- or 10-membered bicyclic heteroaromatic ring is optionally substituted by one or more substituents selected from Me, Cl, F, CN, MeO, NH₂, OH, CO₂Me, SO₂Me, thienyl and fluoropyridinyl.

Preferably, ring A is as defined below, where the wavy lines denote attachment to the ring containing N, Z and Y:

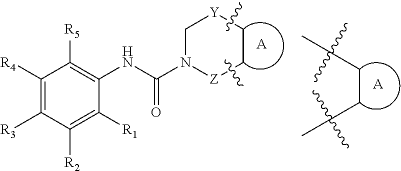

In one preferred embodiment, ring A is selected from:

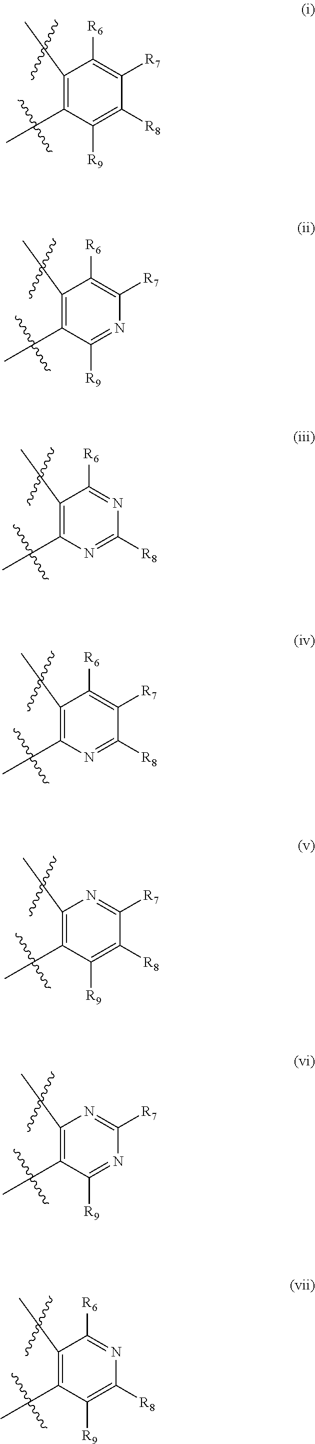

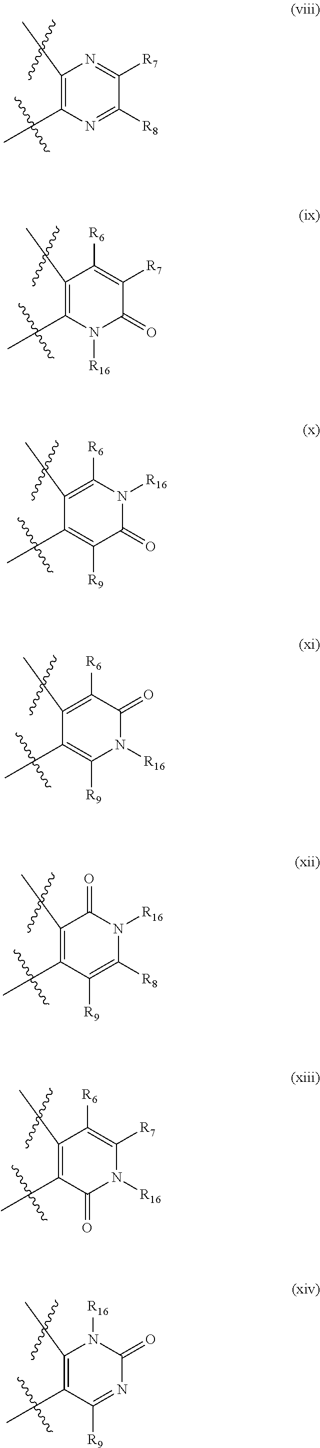

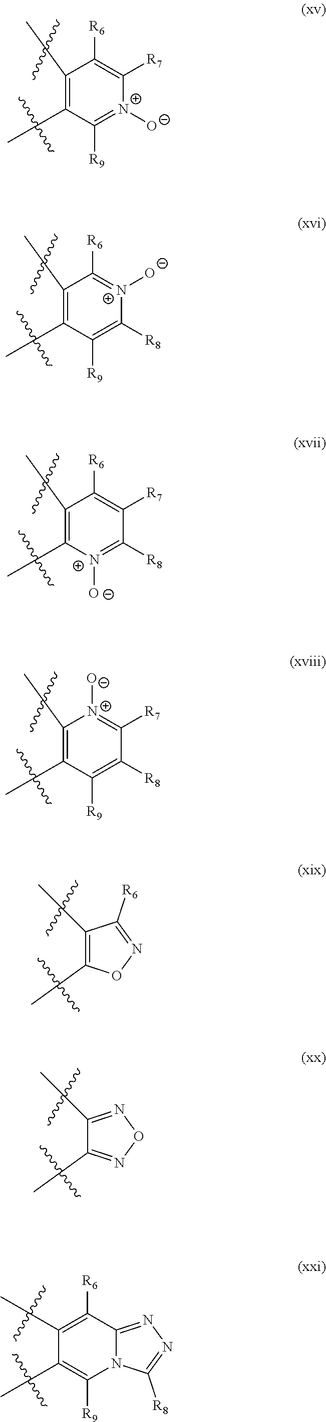

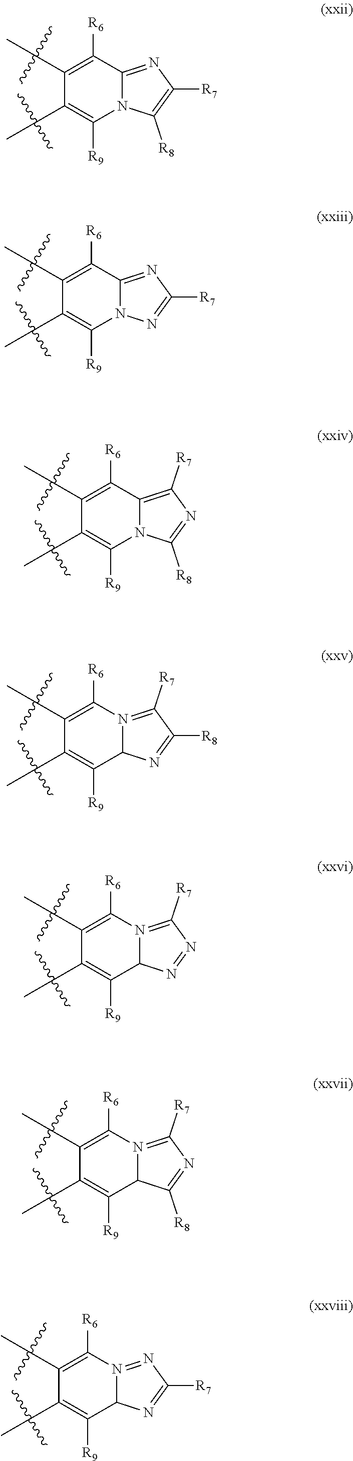

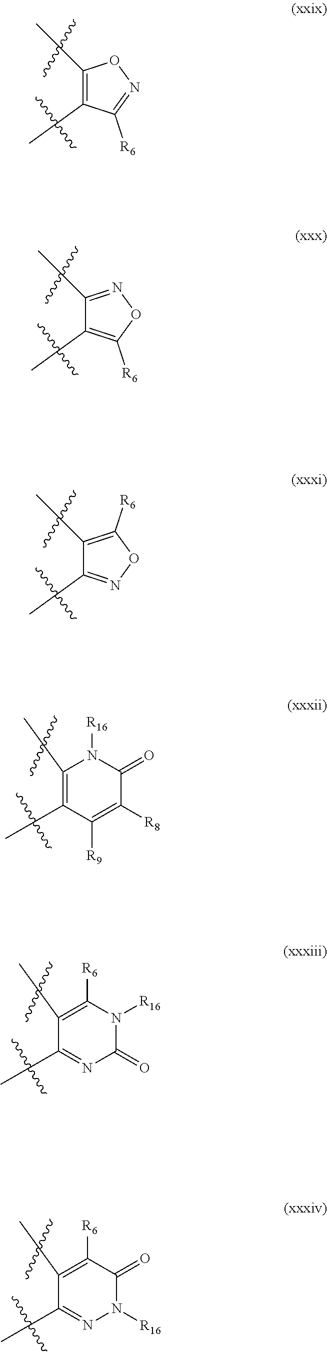

wherein R₆, R₇, R₈, and R₉ are each independently selected from H, halo, CN, alkoxy, CO₂-alkyl, SO₂-alkyl, NR₁₁R₁₁′, OH, alkyl, optionally substituted heteroaryl, phenyl, and haloalkyl, and R₁₆ is H or alkyl.

In one preferred embodiment, R₆, R₇, R₈, and R₉ are each independently selected from H, halo, CN, CO₂-alkyl, SO₂-alkyl, NR₁₁R₁₁′, OH, alkyl, optionally substituted heteroaryl, phenyl, and haloalkyl, and R₁₆ is H or alkyl.

In one embodiment, the compound is other than:
N-(3-fluoro-4-(4-methyl-1H-imidazol-1-yl)phenyl)-6-methoxy-3,4-dihydroisoquinoline-2(1H)-carboxamide;
N-(3-fluoro-4-(4-methyl-1H-imidazol-1-yl)phenyl)-3,4-dihydroisoquinoline-2(1H)-carboxamide;
N-(3-fluoro-4-(4-methyl-1H-imidazol-1-yl)phenyl)-6,7-dimethoxy-3,4-dihydroisoquinoline-2(1H)-carboxamide.

In one preferred embodiment, R₁₂ and R₁₃ are each independently C₁₋₆-alkyl. Even more preferably, R₁₂ and R₁₃ are both Me.

In one preferred embodiment, R₁₁ and R₁₁′ are selected from H and alkyl, and more preferably selected from H and Me, Even more preferably, R₁₁ and R₁₁′ are both H.

In one preferred embodiment, R₁₄, R₁₄′ and R₁₅ are each independently selected from H, and alkyl, more preferably, H and Me.

In one preferred embodiment, ring A is selected from groups (ii)-(xxxiv) defined above.

The following preferred definitions for R₆-R₉ apply to all of the A groups (i)-(xxxiv) defined herein.

In one preferred embodiment, R₆, R₇, R₈, and R₉ are each independently selected from H, F, Cl, Br, I, CN, C₁-C₆ alkoxy, NR₁₁R₁₁′, heteroaryl, OH, C₁-C₆ alkyl, phenyl, and C₁-C₆ haloalkyl, and R₁₆ is H or alkyl.

In one preferred embodiment, R₁₆ is H or Me, more preferably H.

In one preferred embodiment, R₆ is selected from H, F, Cl, CN, methoxy, CH₃, NR₁₁R₁₁′, and CF₃, wherein R₁₁ and R₁₁′ are each independently selected from H and C₁-C₆ alkyl. More preferably R₁₁ and R₁₁′ are both H.

In one preferred embodiment, R₆ is selected from H, F, Cl, CN, methoxy, and CH₃. In one particularly preferred embodiment, R₆ is H or F.

In one preferred embodiment, R₇ is selected from H, F, Cl, CN, methoxy, CH₃, NR₁₁R₁₁′ and CF₃, wherein R₁₁ and R₁₁′ are each independently selected from H and C₁-C₆ alkyl.

In one preferred embodiment, R₇ is selected from H, NH₂, F, Cl, CN, methoxy, CH₃, and CF₃. More preferably R₇ is selected from H, NH₂, F, and Cl. In one particularly preferred embodiment, R₇ is H.

In one preferred embodiment, R₈ is selected from H, F, OH, CN, methoxy, NR₁₁R₁₁′, phenyl, CF₃, CF₂H, NHSO₂CH₃, NHCOCH₃, and NHCHF₂, wherein R₁₁ and R₁₁′ are each independently selected from H and C₁-C₆ alkyl. More preferably R₁₁ and R₁₁′ are both H.

In one preferred embodiment, R₈ is selected from H, F, Cl, CN, methoxy, CH₃, and CF₃, more preferably from H, F, Cl, and CN. In one particularly preferred embodiment, R₈ is H.

In one preferred embodiment, R₉ is selected from H, F, Cl, CN, methoxy, CH₃, NR₁₁R₁₁′ and CF₃, wherein R₁₁ and R₁₁′ are each independently selected from H and C₁-C₆ alkyl, more preferably, wherein R₁₁ and R₁₁′ are both H.

In one preferred embodiment, R₉ is selected from H, F, Cl, CN, methoxy, CH₃, and CF₃, more preferably H, F, and CN. In one particularly preferred embodiment, R₉ is H.

In one preferred embodiment, ring A is selected from groups (i), (ii) and (iv)-(xxxiv).

In one particularly preferred embodiment, ring A is selected from groups (vii), (xi) and (xxxiv).

Thus, preferably the compound is selected from the following formulae:

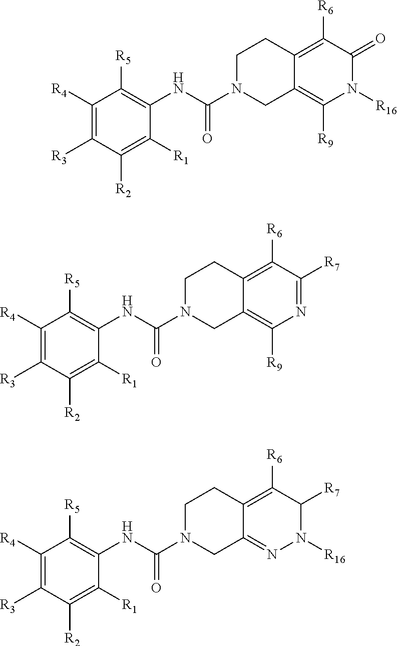

In one preferred embodiment, ring A is (vii) and R₆ is F, and R₈ and R₉ are H.

In one preferred embodiment, ring A is (xi) and R₆, R₉ and R₁₆ are all H.

In one preferred embodiment, ring A is (xxxiv) and R₆ and R₁₆ are both H.

In one preferred embodiment, ring A is:

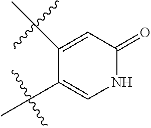 [SMILES: *c1c[nH]c(=O)cc1*]

In one preferred embodiment, the compound is of formula (Ia):

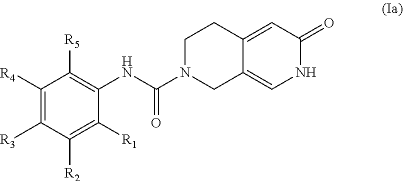

wherein R₁-R₅ are as defined above.

In one preferred embodiment, ring A is:

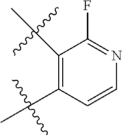 [SMILES: *c1ccnc(F)c1*]

In one preferred embodiment, the compound is of formula (Ib):

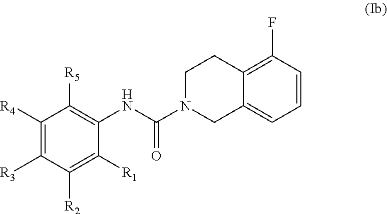

wherein R₁-R₅ are as defined above.

In one preferred embodiment, ring A is:

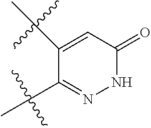 [SMILES: *c1cc(=O)[nH]nc1*]

In one preferred embodiment, the compound is of formula (Ic):

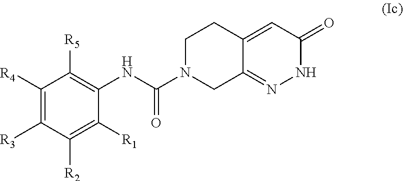

wherein R₁-R₅ are as defined above.

Preferably, when A is a group (i) and R₂ is F, R₁, R₄ and R₅ are all H, R₃ is other than 4-methyl-1H-imidazol-1-yl.

In one preferred embodiment, the compound according to the invention, or for use according to the invention is selected from the following:

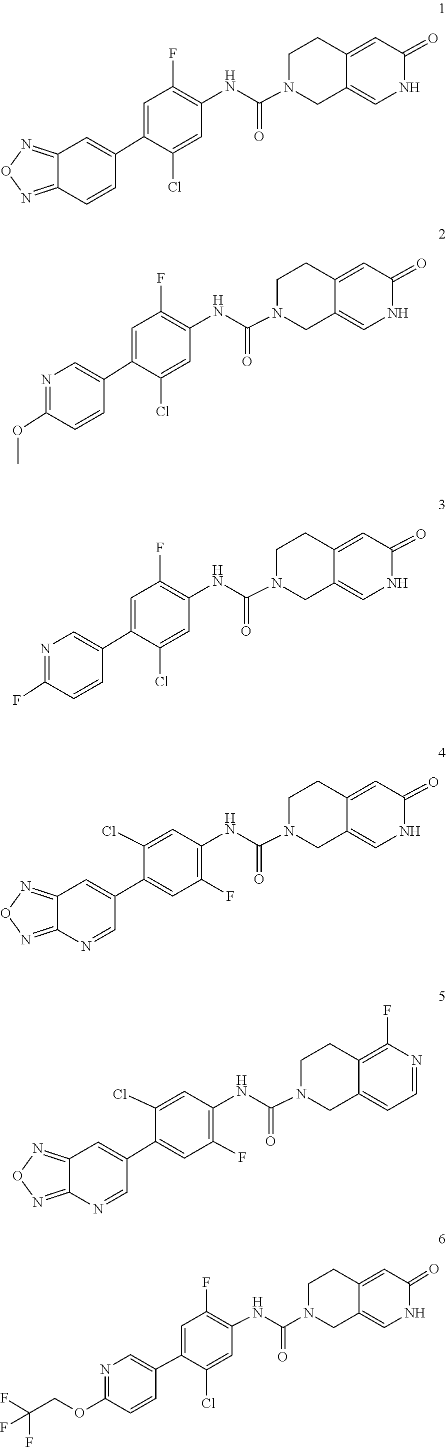 [SMILES: O=C(Nc1cc(Cl)c(-c2ccc3nonc3c2)cc1F)N1CCc2cc(=O)[nH]cc2C1 ; COc1ccc(-c2cc(F)c(NC(=O)N3CCc4cc(=O)[nH]cc4C3)cc2Cl)cn1 ; O=C(Nc1cc(Cl)c(-c2ccc(F)nc2)cc1F)N1CCc2cc(=O)[nH]cc2C1 ; O=C(Nc1cc(Cl)c(-c2cnc3nonc3c2)cc1F)N1CCc2cc(=O)[nH]cc2C1 ; O=C(Nc1cc(Cl)c(-c2cnc3nonc3c2)cc1F)N1CCc2c(ccnc2F)C1 ; O=C(Nc1cc(Cl)c(-c2ccc(OCC(F)(F)F)nc2)cc1F)N1CCc2cc(=O)[nH]cc2C1]

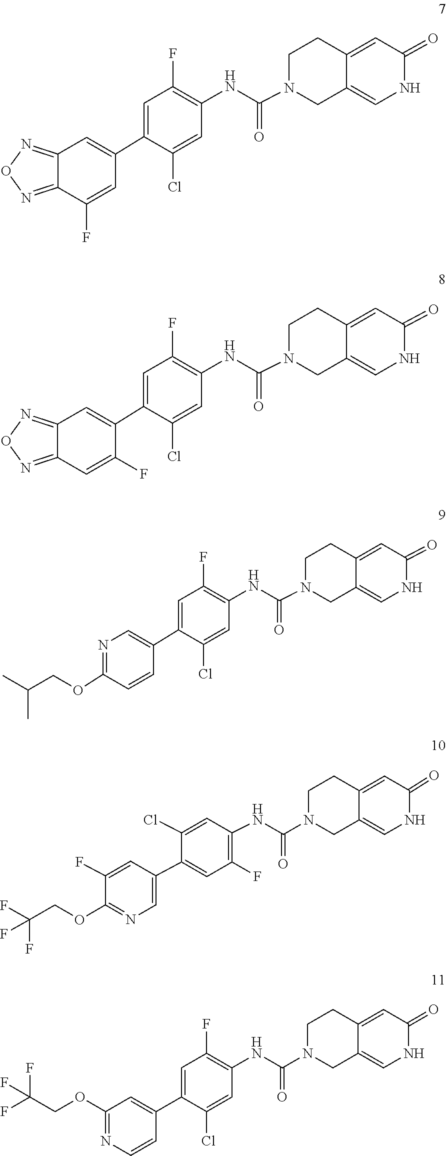 [SMILES: O=C(Nc1cc(Cl)c(-c2cc(F)c3nonc3c2)cc1F)N1CCc2cc(=O)[nH]cc2C1 ; O=C(Nc1cc(Cl)c(-c2cc3nonc3cc2F)cc1F)N1CCc2cc(=O)[nH]cc2C1 ; CC(C)COc1ccc(-c2cc(F)c(NC(=O)N3CCc4cc(=O)[nH]cc4C3)cc2Cl)cn1 ; O=C(Nc1cc(Cl)c(-c2cnc(OCC(F)(F)F)c(F)c2)cc1F)N1CCc2cc(=O)[nH]cc2C1 ; O=C(Nc1cc(Cl)c(-c2ccnc(OCC(F)(F)F)c2)cc1F)N1CCc2cc(=O)[nH]cc2C1]

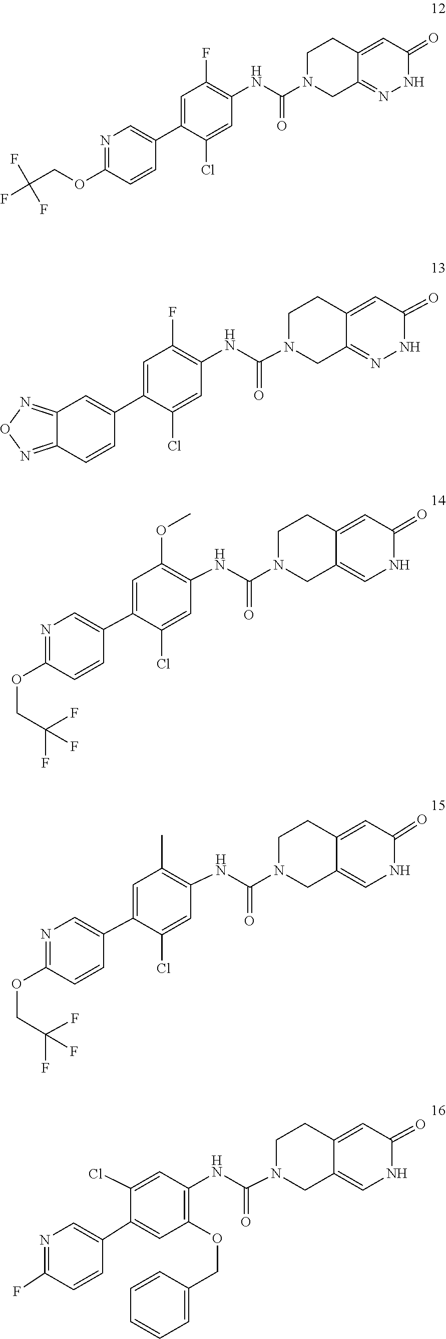 [SMILES: O=C(Nc1cc(Cl)c(-c2ccc(OCC(F)(F)F)nc2)cc1F)N1CCc2cc(=O)[nH]nc2C1 ; O=C(Nc1cc(Cl)c(-c2ccc3nonc3c2)cc1F)N1CCc2cc(=O)[nH]nc2C1 ; COc1cc(-c2ccc(OCC(F)(F)F)nc2)c(Cl)cc1NC(=O)N1CCc2cc(=O)[nH]cc2C1 ; Cc1cc(-c2ccc(OCC(F)(F)F)nc2)c(Cl)cc1NC(=O)N1CCc2cc(=O)[nH]cc2C1 ; O=C(Nc1cc(Cl)c(-c2ccc(F)nc2)cc1OCc1ccccc1)N1CCc2cc(=O)[nH]cc2C1]

and pharmaceutically acceptable salts and solvates thereof.

Preferred compounds according to the invention, or for use according to the invention are alternatively described below:

| | |
|---|---|
| 1 | N-(4-(Benzo[c][1,2,5]oxadiazol-5-yl)-5-chloro-2-fluorophenyl)-6- |
| | oxo-3,4,6,7-tetrahydro-2,7-naphthyridine-2(1H)-carboxamide |
| 2 | N-(5-Chloro-2-fluoro-4-(6-methoxypyridin-3-yl)phenyl)-6-oxo- |
| | 3,4,6,7-tetrahydro-2,7-naphthyridine-2(1H)-carboxamide |
| 3 | N-(5-chloro-2-fluoro-4-(6-fluoropyridin-3-yl)phenyl)-6-oxo-3,4,6,7- |
| | tetrahydro-2,7-naphthyridine-2(1H)-carboxamide |
| 4 | N-(4-([1,2,5]Oxadiazolo[3,4-b]pyridin-6-yl)-5-chloro-2- |
| | fluorophenyl)-6-oxo-3,4,6,7-tetrahydro-2,7-naphthyridine-2(1H)- |
| | carboxamide |
| 5 | N-(4-([1,2,5]Oxadiazolo[3,4-b]pyridin-6-yl)-5-chloro-2- |
| | fluorophenyl)-5-fluoro-3,4-dihydro-2,6-naphthyridine-2(1H)- |
| | carboxamide |
| 6 | N-(5-Chloro-2-fluoro-4-(6-(2,2,2-trifluoroethoxy)pyridin-3- |
| | yl)phenyl)-6-oxo-3,4,6,7-tetrahydro-2,7-naphthyridine-2(1H)- |
| | carboxamide |
| 7 | N-(5-Chloro-2-fluoro-4-(7-fluorobenzo[c][1,2,5]oxadiazol-5- |
| | yl)phenyl)-6-oxo-3,4,6,7-tetrahydro-2,7-naphthyridine-2(1H)- |
| | carboxamide |
| 8 | N-(5-Chloro-2-fluoro-4-(6-fluorobenzo[c][1,2,5]oxadiazol-5- |
| | yl)phenyl)-6-oxo-3,4,6,7-tetrahydro-2,7-naphthyridine-2(1H)- |
| | carboxamide |
| 9 | N-(5-Chloro-2-fluoro-4-(6-isobutoxypyridin-3-yl)phenyl)-6-oxo- |
| | 3,4,6,7-tetrahydro-2,7-naphthyridine-2(1H)-carboxamide |
| 10 | N-(5-Chloro-2-fluoro-4-(5-fluoro-6-(2,2,2-trifluoroethoxy)pyridin-3- |
| | yl)phenyl)-6-oxo-3,4,6,7-tetrahydro-2,7-naphthyridine-2(1H)- |
| | carboxamide |
| 11 | N-(5-Chloro-2-fluoro-4-(2-(2,2,2-trifluoroethoxy)pyridin-4- |
| | yl)phenyl)-6-oxo-3,4,6,7-tetrahydro-2,7-naphthyridine-2(1H)- |
| | carboxamide |
| 12 | N-(5-Chloro-2-fluoro-4-(6-(2,2,2-trifluoroethoxy)23yridine-3- |
| | yl)phenyl)-3-oxo-2,5,6,8-tetrahydropyrido[3,4-c]pyridazine-7(3H)- |
| | carboxamide |
| 13 | N-(4-(Benzo[c][1,2,5]oxadiazol-5-yl)-5-chloro-2-fluorophenyl)-3- |
| | oxo-2,5,6,8-tetrahydropyrido[3,4-c]pyridazine-7(3H)-carboxamide |
| 14 | N-(5-chloro-2-methoxy-4-(6-(2,2,2-trifluoroethoxy)pyridin-3- |
| | yl)phenyl)-6-oxo-3,4,6,7-tetrahydro-2,7-naphthyridine-2(1H)- |
| | carboxamide |
| 15 | N-(5-Chloro-2-methyl-4-(6-(2,2,2-trifluoroethoxy)pyridin-3- |
| | yl)phenyl)-6-oxo-3,4,6,7-tetrahydro-2,7-naphthyridine-2(1H)- |
| | carboxamide |
| 16 | N-(2-(Benzyloxy)-5-chloro-4-(6-fluoropyridin-3-yl)phenyl)-6-oxo- |
| | 3,4,6,7-tetrahydro-2,7-naphthyridine-2(1H)-carboxamide |

and pharmaceutically acceptable salts and solvates thereof.

In one preferred embodiment, the compound according to the invention, or for use according to the invention is selected from the following: Compounds 1-2 and 5-13.

### Process

A further aspect of the invention relates to a process for preparing compounds as defined herein, said process comprising reacting a compound of formula (II) with a compound of formula (III), where R¹⁻⁵, Z, Y and A are as defined above, in the presence of triphosgene to form a compound of formula (I) or (I′):

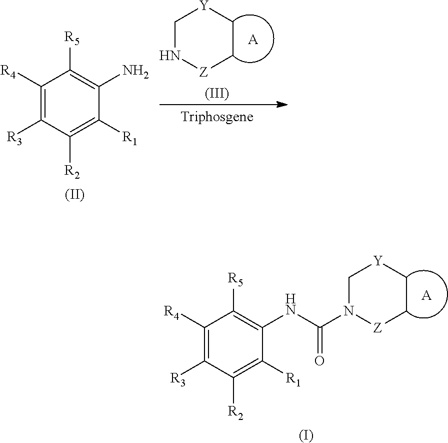

In one preferred embodiment, the reaction takes place in the presence of a base, preferably, N,N-diisopropylethylamine (DIPEA) or trimethylamine or dimethylaminopyridine (DMAP). Preferably, the reaction takes place in an organic solvent. Suitable organic solvents include, but are not limited to, dichloromethane, tetrahydrofuran and dimethylformamide, or mixtures of two or more thereof. The skilled person would understand that other bases and solvents would also be suitable.

### Therapeutic Applications

A further aspect of the invention relates to compounds as described herein for use in medicine. The compounds have particular use in the field of oncology, immuno-oncology, and immunology as described in more detail below. In a preferred embodiment, the compound of the invention modulates GPR65, and more preferably inhibits GPR65 signalling.

Yet another aspect of the invention relates to compounds as described herein for use as a medicament.

Preferably, the compounds according to the invention are for use in treating or preventing a disease or disorder selected from a proliferative disorder, an autoimmune disorder, asthma and chronic obstructive pulmonary disease.

One preferred embodiment of the invention relates to compounds as described herein for use in treating or preventing a disorder selected from a proliferative disorder and an immune disorder.

Another preferred embodiment of the invention relates to compounds as described herein for use in treating or preventing asthma and/or chronic obstructive pulmonary disease (COPD). GPR65 variant/SNP (rs6574978) has been shown to be associated with asthma/COPD syndrome with almost GWAS significant p value (1.18×10e-7) (Hardin 2014).

Furthermore, GPR65 activation by pH (pH is low/acidic in asthmatic lungs) promotes eosinophil viability in a cAMP-dependent manner, contributing to disease progression/exacerbation. It is further known that GPR65 KO mice have attenuated asthma symptoms (Kottyan 2009).

Another aspect of the invention relates to compounds as described herein for use in treating or preventing acute respiratory distress syndrome (ARDS). GPR65 has been shown to be protective in a model of LPS-induced acute lung injury model (Tsurumaki 2015).

One aspect of the invention relates to a compound as described herein for use in treating a proliferative disorder. Preferably, the proliferative disorder is a cancer or leukemia.

In one preferred embodiment, the cancer is a solid tumour and/or metastases thereof.

In another preferred embodiment, the cancer is selected from melanoma, renal cell carcinoma (RCC), gastric cancer, acute myeloid leukaemia (AML), pancreatic adenocarcinoma, triple negative breast cancer (TNBC), colorectal cancer, head and neck cancer, colorectal adenocarcinoma, lung cancer, sarcoma, ovarian cancer, and gliomas, preferably glioblastoma (GBM).

Without wishing to be bound by theory, it is understood that GPR65 modulators are capable of preventing the increase in cytoplasmic cAMP in tumour-associated macrophages (TAMs), natural killer (NK) cells and subsets of T cells that would typically result from their exposure to the acidic tumour microenvironment and concomitant GPR65 activation. This reduction in the level of cytoplasmic cAMP in turn reduces the levels of ICER pro-inflammatory mediators such as CXCL10 and TNFα, preventing the polarization of TAMs and alteration of other immune cells that are associated with a non-inflammatory and tumour-permissive environment. Therefore, GPR65 modulators are expected to result in an increase in the visibility of the tumour to the immune system leading to increased immune-mediated tumour clearance. This suggests that modulation of GPR65 activity could be an effective treatment for cancer as stand-alone therapy or in combination with cancer immunotherapies (vaccines, agents that promote T cell mediated immune responses) or in patients that do not respond to immunomodulatory approaches such as PD1/PDL-1 blockade.

Another aspect of the invention relates to a compound as described herein for use in treating an immune disorder, preferably an autoimmune disease.

In one embodiment, the autoimmune disease is selected from psoriasis, psoriatic arthritis, rheumatoid arthritis (RA), multiple sclerosis (MS), systemic lupus erythematosus (SLE), autoimmune thyroiditis (Hashimoto's thyroiditis), Graves' disease, uveitis (including intermediate uveitis), ulcerative colitis, Crohn's disease, autoimmune uveoretinitis, systemic vasculitis, polymyositis-dermatomyositis, systemic sclerosis (scleroderma), Sjogren's Syndrome, ankylosing spondylitis and related spondyloarthropathies, sarcoidosis, autoimmune hemolytic anemia, immunological platelet disorders, autoimmune polyendocrinopathies and autoimmune myocarditis, type I diabetes and atopic dermatitis

In a particularly preferred embodiment, the autoimmune disease is selected from psoriasis, psoriatic arthritis, ankylosing spondylitis, Crohn's disease, and multiple sclerosis (MS).

Without wishing to be bound by theory, it is understood that GPR65 modulators will prevent the upregulation of ICER in CD4+ T cells. This, in turn, is expected to prevent the ICER-associated suppression of IL-2 that biases CD4+ T cells toward the inflammatory Th17 phenotype associated with increased pathogenicity in the context of autoimmune disease. This is supported by the fact that mutations in the GPR65 locus are associated with several autoimmune diseases, such as multiple sclerosis, ankylosing spondylitis, inflammatory bowel disease, and Crohn's disease (Gaublomme, 2015). This suggests that modulation of GPR65 activity could be an effective treatment for autoimmune diseases.

Another aspect relates to a compound as described herein for use in treating or preventing a disorder caused by, associated with or accompanied by abnormal activity against GPR65.

Another aspect relates to a compound as described herein for use in treating or preventing a GPR65-associated disease or disorder.

Another aspect of the invention relates to a method of treating a disorder as described above comprising administering a compound as described herein to a subject.

Another aspect of the invention relates to a method of treating a GPR65-associated disease or disorder in a subject. The method according to this aspect of the present invention is effected by administering to a subject in need thereof a therapeutically effective amount of a compound of the present invention, as described hereinabove, either per se, or, more preferably, as a part of a pharmaceutical composition, mixed with, for example, a pharmaceutically acceptable carrier, as is detailed hereinafter.

Yet another aspect of the invention relates to a method of treating a subject having a disease state alleviated by modulation of GPR65 wherein the method comprises administering to the subject a therapeutically effective amount of a compound according to the invention.

Another aspect relates to a method of treating a disease state alleviated by modulation of GPR65, wherein the method comprises administering to a subject a therapeutically effective amount of a compound according to the invention.

Preferably, the subject is a mammal, more preferably a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₁₀₀ as determined in cell culture. Such information can be used to more accurately determine useful doses in humans. Initial dosages can also be estimated from in vivo data. Using these initial guidelines one of ordinary skill in the art could determine an effective dosage in humans.

Moreover, toxicity and therapeutic efficacy of the compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (see, e.g., Fingl et al, 1975, The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/day, commonly from about 100-1000 mg/day, preferably from about 150-700 mg/day and most preferably from about 250-500 mg/day, or from 50-100 mg/day. Preferably, therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

As used herein, “GPR65-related disease or disorder” refers to a disease or disorder characterized by inappropriate GPR65 activity. Inappropriate GPR65 activity refers to either an increase or decrease in GPR65 activity as measured by enzyme or cellular assays, for example, compared to the activity in a healthy subject. Inappropriate activity could also be due to overexpression of GPR65 in diseased tissue compared with healthy adjacent tissue.

Preferred diseases or disorders that the compounds described herein may be useful in treating or preventing include proliferative disorders and immune disorders as described hereinbefore, as well as asthma and chronic obstructive pulmonary disease.

The present invention further provides for the use of compounds as defined herein in the preparation of a medicament for the treatment of a disease where it is desirable to modulate GPR65. Such diseases include proliferative disorders and immune disorders as described hereinbefore, as well as asthma and chronic obstructive pulmonary disease.

As used herein the phrase “preparation of a medicament” includes the use of the components of the invention directly as the medicament in addition to their use in any stage of the preparation of such a medicament.

In one preferred embodiment, the compound prevents the increase in cytoplasmic cAMP levels expected following GPR65 activation at acidic pH. This prevention of cAMP accumulation is expected in turn to prevent the undesirable downstream signalling through ICER, as described in the accompanying examples section. The “Human GPR65 cyclic adenosine monophosphate (cAMP) Homogeneous Time Resolved Fluorescence (HTRF) antagonist assay”, or simply “cAMP assay”, as described below, can be used to measure the potency of GPR65 modulators, which is expressed as the concentration of compound required to reduce the increase in cAMP concentration upon GPR65 activation by 50% (i.e. an IC₅₀).

In one preferred embodiment, the compound exhibits an IC₅₀ value in the cAMP assay of less than about 25 μM. More preferably, the compound exhibits an IC₅₀ value in the cAMP assay of less than about 10 μM, more preferably, less than about 5 μM, even more preferably, less than about 1 μM, even more preferably, less than about 0.1 μM.

In another preferred embodiment, the compound exhibits an hGPR65 IC₅₀ value of less than <5 μM, more preferably less than <500 nM in the aforementioned assay.

### Pharmaceutical Compositions

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers, diluents or excipients therefor and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller. The carrier, or, if more than one be present, each of the carriers, must be acceptable in the sense of being compatible with the other ingredients of the formulation and not deleterious to the recipient.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound as described herein into conjunction or association with a pharmaceutically or veterinarily acceptable carrier or vehicle.

### Salts/Esters

The compounds of the invention can be present as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

The present invention also includes all suitable isotopic variations of the compound or a pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formula (I) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Atropisomers

Some of the compounds of the invention may exist as atropisomers. Atropisomers are stereoisomers arising because of hindered rotation about a single bond, where energy differences due to steric strain or other contributors create a barrier to rotation that is high enough to allow for isolation of individual conformers. The invention encompasses all such atropisomers. The invention also encompasses rotamers of the compounds.

### Prodrugs

The invention further includes the compounds of the present invention in prodrug form, i.e. covalently bonded compounds which release the active parent drug in vivo. Such prodrugs are generally compounds of the invention wherein one or more appropriate groups have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Solvates

The present invention also includes solvate forms of the compounds of the present invention. The terms used in the claims encompass these forms. Preferably the solvate is a hydrate.

### Combinations

A further aspect of the invention relates to a combination comprising a compound as described herein and one or more additional active agents. In a particularly preferred embodiment, the one or more compounds of the invention are administered in combination with one or more additional active agents, for example, existing drugs available on the market. In such cases, the compounds of the invention may be administered consecutively, simultaneously or sequentially with the one or more other active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance.

Beneficial combinations may be suggested by studying the activity of the test compounds with agents known or suspected of being valuable in the treatment of a particular disorder. This procedure can also be used to determine the order of administration of the agents, i.e. before, simultaneously, or after delivery. Such scheduling may be a feature of all the active agents identified herein.

In the context of cancer, compounds of the invention can be used in combination with immunotherapies such as cancer vaccines and/or with other immune-modulators such as agents that block the PD1/PDL-1 interaction. Other examples of agents for use in combination with the presently claimed compounds include immune modulators that block CTLA-4 or LAG-3. Thus, in one preferred embodiment, the additional active agent is an immunotherapy agent, more preferably a cancer immunotherapy agent. An “immunotherapy agent” refers to a treatment that uses the subject's own immune system to fight diseases such as cancer. For other disorders the compounds of the invention can be used in combination agents that block or decrease inflammation such as antibodies that target pro-inflammatory cytokines. The compounds of the invention can also be used in combination with other chemotherapy agents and/or in conjunction with radiotherapy.

### Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

The dosage amount will further be modified according to the mode of administration of the compound. For example, to achieve an “effective amount” for acute therapy, parenteral administration of a compound is typically preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg; preferably between 0.1 and 20 mg, in a manner to maintain the concentration of drug in the plasma at a concentration effective to modulate GPR65. The compounds may be administered one to four times daily at a level to achieve a total daily dose of about 0.4 to about 400 mg. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 500 mg or about 0.1 to about 50 mg in a manner consistent with the condition of the patient. Preferably the oral dose would be about 0.5 to about 50 mg or about 0.5 to about 20 mg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

The invention is further described by the way of the following non-limiting examples.

### EXAMPLES

Where the preparation of starting materials is not described, these are commercially available, known in the literature, or readily obtainable by those skilled in the art using standard procedures. Where it is indicated that compounds were prepared analogously to earlier examples or intermediates, it will be appreciated by the skilled person that the reaction time, number of equivalents of reagents, solvent, concentration and temperature can be modified for each specific reaction and that it may be necessary or desirable to employ different work-up or purification techniques.

### General Schemes

### Abbreviations

A list of some common abbreviations is shown below—where other abbreviations are used which are not listed, these will be understood by the person skilled in the art.

br.: broad; d: doublet; DCM: dichloromethane; dppf: 1,1′-Bis(diphenylphosphino)ferrocene; DMSO: dimethylsulfoxide; (ES+): electrospray ionization positive mode; Et₃N: triethylamine; EtOAc: ethyl acetate; EtOH: ethanol; h: hours; HPLC: high performance liquid chromatography; Hz: hertz; J: coupling constant; l: litre; M: molar; m: multiplet; [M+H]+: protonated molecular ion; MeCN: acetonitrile; MeOH: methanol; MHz: megahertz; min: minutes; ml: millilitres; MS: mass spectrometry; m/z: mass-to-charge ratio; NMR: nuclear magnetic resonance; PDA: photodiode array; PIDA: (diacetoxyiodo)benzene; RT: room temperature; Rt: retention time; s: singlet; t: triplet; THF: tetrahydrofuran; TLC: thin layer chromatography; UPLC: ultra performance liquid chromatography; UV: ultra-violet.

Other abbreviations are intended to convey their generally accepted meaning.

### General Experimental Conditions

All starting materials and solvents were obtained either from commercial sources or prepared according to literature methods. The appropriate isocyanate and aniline starting materials were obtained from Sigma Aldrich, Fluorochem or Enamine store, or were synthesised as described herein. Reaction mixtures were magnetically stirred and reactions performed at room temperature (approximately 20° C.) unless otherwise indicated.

Silica gel chromatography was performed on an automated flash chromatography system, such as CombiFlash Companion, CombiFlash Rf system or Reveleris X2 flash system using RediSep® Rf or Reveleris® or the GraceResolv™ pre-packed silica (230-400 mesh, 40-63 μm) cartridges.

Analytical UPLC-MS experiments to determine retention times and associated mass ions were performed using a Waters ACQUITY UPLC® H-Class system, equipped with ACQUITY PDA Detector and ACQUITY QDa mass spectrometer or Waters SQD mass spectrometer, running the analytical method described below.

Preparative HPLC purifications were performed either using a Waters Xbridge Prep OBD C18, 10 μm, 40×150 mm column using a gradient of MeCN and 0.1% ammonia in water or a gradient of MeCN and 0.1% formic acid in water. Fractions were collected following UV detection across all wavelengths with PDA and in some cases an SQD2 or ACQUITY QDa mass spectrometer.

Preparative HPLC purifications were performed either using a Waters Xbridge Prep OBD C18, 10 μm, 40×150 mm column using a gradient of MeCN and 0.1% ammonia in water or a gradient of MeCN and 0.1% formic acid in water. Fractions were collected following UV detection across all wavelengths with PDA and in some cases an SQD2 or ACQUITY QDa mass spectrometer.

NMR spectra were recorded using either a Bruker Avance III HD 500 MHz instrument, a Bruker Avance Neo 400 MHz, Bruker Avance III 400 MHz instrument or a QOne AS400 400 MHz spectrometer using either residual non-deuterated solvent, or tetra-methylsilane as a reference

### Analytical Methods

### Method 1—Basic 3 min Method

Column: Waters ACQUITY UPLC® BEH C18, 1.7 μm, 2.1×30 mm at 40° C.
Detection: UV at 210-400 nm unless otherwise indicated, MS by electrospray ionisation
Solvents: A: 0.1% Ammonia in water, B: MeCN
Gradient:

| Time | % A | % B | Flow rate (ml/min) |
|---|---|---|---|
| 0.00 | 98 | 2 | 0.77 |
| 2.50 | 0 | 100 | 0.77 |
| 3.00 | 0 | 100 | 0.77 |

### Method 2—Acidic 3 min Method 1

Column: Waters CSH C18, 1.7 μm, 2.1×30 mm at 40° C.
Detection: UV at 210-400 nm unless otherwise indicated, MS by electrospray ionisation
Solvents: A: 0.1% formic acid in water, B: MeCN
Gradient:

| Time | % A | % B | Flow rate (ml/min) |
|---|---|---|---|
| 0.00 | 98 | 2 | 0.77 |
| 2.5 | 0 | 100 | 0.77 |
| 3 | 0 | 100 | 0.77 |

### Method 3—Acidic 5 min Method 1

Column: Agilent EclipsePlus RRHD C18, 1.8 μm, 3.0×50 mm at 25° C.
Detection: UV at 214 and 254 nm unless otherwise indicated, MS by electrospray ionisation
Solvents: A: 0.05% formic acid in water, B: 0.05% formic acid in MeCN
Gradient:

| Time | % A | % B | Flow rate (ml/min) |
|---|---|---|---|
| 0.00 | 90 | 10 | 0.8 |
| 0.50 | 90 | 10 | 0.8 |
| 4.00 | 10 | 90 | 0.8 |
| 4.50 | 0 | 100 | 0.8 |
| 4.51 | 90 | 10 | 0.8 |
| 5.00 | 90 | 10 | 0.8 |

### Method 4—Acidic 5 min Method 2

Column: Waters Sunfire, 3.5 μm, 4.6×50 mm column at 25° C.
Detection: UV at 214 and 254 nm unless otherwise indicated, MS by electrospray ionisation
Solvents: A: 0.05% formic acid in water, B: 0.05% formic acid in MeCN
Gradient:

| Time | % A | % B | Flow rate (ml/min) |
|---|---|---|---|
| 0.00 | 85 | 15 | 1.0 |
| 0.50 | 85 | 15 | 1.0 |
| 4.00 | 0 | 100 | 1.0 |
| 4.50 | 0 | 100 | 1.0 |
| 4.51 | 85 | 15 | 1.0 |
| 5.00 | 85 | 15 | 1.0 |

### Method 5—Acidic 3 min Method 2

Column: Waters CORTECS UPLC, C18, 1.6 μm, 2.1×50 mm column at 25° C.
Detection: UV at 210-400 nm unless otherwise indicated, MS by electrospray ionisation
Solvents: A: 0.1% formic acid in water, B: MeCN
Gradient:

| Time | % A | % B | Flow rate (ml/min) |
|---|---|---|---|
| 0.00 | 80 | 20 | 0.6 |
| 1.80 | 20 | 80 | 0.6 |
| 2.65 | 20 | 80 | 0.6 |
| 2.80 | 80 | 20 | 0.6 |
| 3.00 | 80 | 20 | 0.6 |

### Experimental Scheme 1

### Compound 1: N-(4-(Benzo[c][1,2,5]oxadiazol-5-yl)-5-chloro-2-fluorophenyl)-6-oxo-3,4,6,7-tetrahydro-2,7-naphthyridine-2(1H)-carboxamide

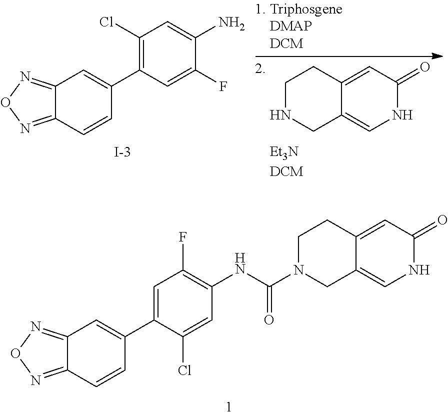

To a solution of triphosgene (16.2 mg, 54.6 μmol) in DCM (1.5 ml) was added a solution of 4-(benzo[c][1,2,5]oxadiazol-5-yl)-5-chloro-2-fluoroaniline I-3 (36.0 mg, 137 μmol) and DMAP (50.0 mg, 410 μmol) in DCM (1.5 ml) and the resulting mixture was stirred at RT for 5 min. To a separate solution of 5,6,7,8-tetrahydro-2,7-naphthyridin-3(2H)-one, HBr (31.6 mg, 137 μmol) in DCM (1.5 ml) was added Et₃N (57 μl, 410 μmol) slowly and the solution was stirred vigorously until the solid had fully dissolved. To this solution was added the initial solution and the resultant white suspension was stirred for 16 h at RT. The reaction mixture was concentrated under reduced pressure. The product was purified by mass directed prep HPLC (35-65% MeCN/(0.1% formic acid in water) on an Waters X-Select CSH C18 ODB prep column, 130 Å, 5 μm, 30 mm×100 mm, flow rate 40 mL min-1, 8.5 min run) to give N-(4-(Benzo[c][1,2,5]oxadiazol-5-yl)-5-chloro-2-fluorophenyl)-6-oxo-3,4,6,7-tetrahydro-2,7-naphthyridine-2(1H)-carboxamide 1 as a colourless solid. LCMS (Method 1) m/z 440.2, 442.2 (M+H)⁺ (ES⁺), at 1.27 min. ¹H NMR (400 MHz, DMSO-d6) δ 11.41 (s, 1H), 8.55 (s, 1H), 8.15 (d, J=1.3 Hz, 1H), 8.14 (dd, J=9.4, 1.1 Hz, 1H), 7.84 (d, J=7.2 Hz, 1H), 7.72 (dd, J=9.3, 1.5 Hz, 1H), 7.55 (d, J=11.0 Hz, 1H), 7.34 (s, 1H), 6.21 (s, 1H), 4.41 (s, 2H), 3.62 (t, J=6.1 Hz, 2H), 2.79 (t, J=6.1 Hz, 2H).

The following compounds were prepared using appropriate starting materials in an analogous procedure to that described in Experimental Scheme 1. Where the starting materials are not described in the literature, their synthesis is described below.

| | | LCMS | |
|---|---|---|---|
| Compound | Structure | Rt [M + H]⁺ | NMR |
| 2^{(b)} | 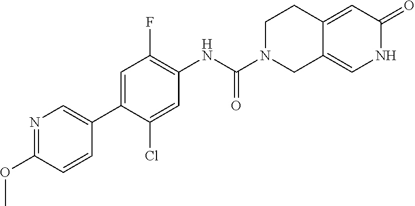 [SMILES: COc1ccc(-c2cc(F)c(NC(=O)N3CCc4cc(=O)[nH]cc4C3)cc2Cl)cn1] | Method 1 429.1, 431.3 at 1.14 min | ¹H NMR (400 MHz, DMSO- d6) δ 8.24 (d, J = 2.5 Hz, 1H), 7.82 (dd, J= 8.6, 2.5 Hz, 1H), 7.77 (d, J = 7.4 Hz, 1H), 7.36 (d, J = 11.2 Hz, 1H), 7.33 (s, 1H), 6.91 (d, J = 8.6 Hz, 1H), 6.20 (s, 1H), 4.40 (s, 2H), 3.90 (s, 3H), 3.61 (t, J = 6.1 Hz, 2H), 2.77 (t, J = 6.1 Hz, 2H). (Two exchangeable protons not observed) |
| | N-(5-Chloro-2-fluoro-4-(6- | | |
| | methoxypyridin-3-yl)phenyl)-6-oxo- | | |
| | 3,4,6,7-tetrahydro-2,7- | | |
| | naphthyridine-2(1H)-carboxamide | | |
| 3^{(b)} | 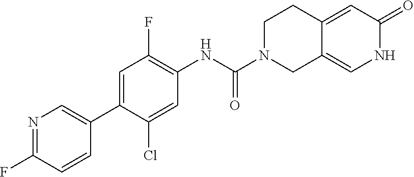 [SMILES: O=C(Nc1cc(Cl)c(-c2ccc(F)nc2)cc1F)N1CCc2cc(=O)[nH]cc2C1] N-(5-chloro-2-fluoro-4-(6- | Method 1 417.3, 419.6 at 1.07 min | ¹H NMR (400 MHz, DMSO- d6) δ 8.33 (d, J = 2.6 Hz, 1H), 8.12 (td, J = 8.2, 2.6 Hz, 1H), 7.81 (d, J = 7.3 Hz, 1H), 7.45 (d, J = 11.1 Hz, 1H), 7.33 (s, 1H), 7.30 (dd, J = 8.5, 2.8 Hz, 1H), 6.21 (s, 1H), 4.40 (s, 2H), 3.61 (t, J = 6.1 Hz, 2H), 2.78 (t, J = 6.1 Hz, 2H). (Two exchangeable protons not observed) |
| | fluoropyridin-3-yl)phenyl)-6-oxo- | | |
| | 3,4,6,7-tetrahydro-2,7- | | |
| | naphthyridine-2(1H)-carboxamide | | |
| 4^{(b)} | 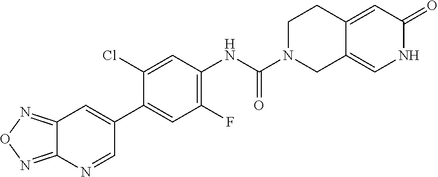 [SMILES: O=C(Nc1cc(Cl)c(-c2cnc3nonc3c2)cc1F)N1CCc2cc(=O)[nH]cc2C1] | Method 2 441.3, 443.2 at 1.47 min | ¹H NMR (400 MHz, DMSO- d6) δ 11.41 (s, 1H), 9.21 (d, J = 2.0 Hz, 1H), 8.73 (d, J = 2.1 Hz, 1H), 8.61 (s, 1H), 7.90 (d, J = 7.2 Hz, 1H), 7.65 (d, J = 11.0 Hz, 1H), 7.34 (s, 1H), 6.21 (s, 1H), 4.42 (s, 2H), 3.63 (t, J= 6.1 Hz, 2H), 2.79 (t, J = 6.1 Hz, 2H). |
| | N-(4-([1,2,5]Oxadiazolo[3,4- | | |
| | b]pyridin-6-yl)-5-chloro-2- | | |
| | fluorophenyl)-6-oxo-3,4,6,7- | | |
| | tetrahydro-2,7-naphthyridine- | | |
| | 2(1H)-carboxamide | | |
| 5 | 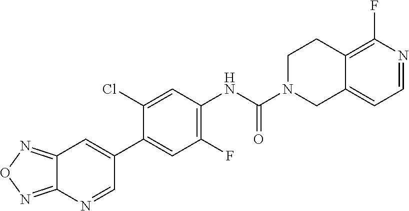 [SMILES: O=C(Nc1cc(Cl)c(-c2cnc3nonc3c2)cc1F)N1CCc2c(ccnc2F)C1] | Method 2 443.0, 445.0 at 1.91 min | ¹H NMR (400 MHz, DMSO- d6) δ 9.21 (d, J = 2.1 Hz, 1H), 8.87 - 8.66 (m, 2H), 8.03 (d, J = 5.0 Hz, 1H), 7.88 (d, J = 7.2 Hz, 1H), 7.65 (d, J = 11.1 Hz, 1H), 7.23 (d, J = 5.2 Hz, 1H), 4.76 (s, 2H), 3.81 (t, J = 5.9 Hz, 2H), 2.81 (t, J = 5.9 Hz, 2H). |
| | N-(4-([1,2,5]Oxadiazolo[3,4- | | |
| | b]pyridin-6-yl)-5-chloro-2- | | |
| | fluorophenyl)-5-fluoro-3,4-dihydro- | | |
| | 2,6-naphthyridine-2(1H)- | | |
| | carboxamide | | |
| 6^{(c)} | 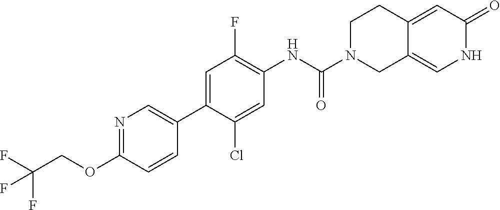 [SMILES: O=C(Nc1cc(Cl)c(-c2ccc(OCC(F)(F)F)nc2)cc1F)N1CCc2cc(=O)[nH]cc2C1] | Method 1 497.1, 499.2 at 1.43 min | ¹H NMR (400 MHz, DMSO- d6) δ 11.41 (s, 1H), 8.48 (s, 1H), 8.28 (dd, J = 2.4, 0.8 Hz, 1H), 7.93 (dd, J = 8.5, 2.5 Hz, 1H), 7.77 (d, J = 7.3 Hz, 1H), 7.42 (d, J = 11.1 Hz, 1H), 7.33 (s, 1H), 7.09 (dd, J = 8.6, 0.7 Hz, 1H), 6.21 (s, 1H), 5.05 (q, J = 9.1 Hz, 2H), 4.40 (s, 2H), 3.61 (t, J = 6.1 Hz, 2H), 2.78 (t, J = 6.1 Hz, 2H). |
| | N-(5-Chloro-2-fluoro-4-(6-(2,2,2- | | |
| | trifluoroethoxy)pyridin-3-yl)phenyl)- | | |
| | 6-oxo-3,4,6,7-tetrahydro-2,7- | | |
| | naphthyridine-2(1H)-carboxamide | | |
| 7^{(c)} | 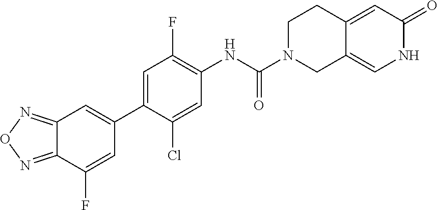 [SMILES: O=C(Nc1cc(Cl)c(-c2cc(F)c3nonc3c2)cc1F)N1CCc2cc(=O)[nH]cc2C1] | Method 1 458.1, 460.1 at 1.33 min | ¹H NMR (400 MHz, DMSO- d6) δ 11.42 (s, 1H), 8.57 (s, 1H), 8.08 (d, J = 1.0 Hz, 1H), 7.85 (d, J = 7.2 Hz, 1H), 7.71 (dd, J = 11.3, 1.0 Hz, 1H), 7.55 (d, J = 11.0 Hz, 1H), 7.34 (s, 1H), 6.21 (s, 1H), 4.41 (s, 2H), 3.62 (t, J = 6.1 Hz, 2H), 2.79 (t, J = 6.1 Hz, 2H). |
| | N-(5-Chloro-2-fluoro-4-(7- | | |
| | fluorobenzo[c][1,2,5]oxadiazol-5- | | |
| | yl)phenyl)-6-oxo-3,4,6,7- | | |
| | tetrahydro-2,7-naphthyridine- | | |
| | 2(1H)-carboxamide | | |
| 8^{(c)} | 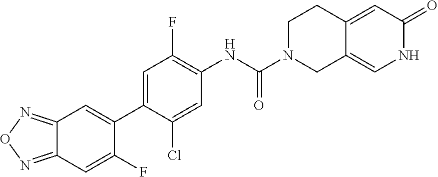 [SMILES: O=C(Nc1cc(Cl)c(-c2cc3nonc3cc2F)cc1F)N1CCc2cc(=O)[nH]cc2C1] | Method 1 458.2, 460.2 at 1.26 min | ¹H NMR (400 MHz, DMSO- d6) δ 11.41 (s, 1H), 8.57 (s, 1H), 8.33 (d, J = 6.5 Hz, 1H), 8.16 - 8.10 (m, 1H), 7.84 (d, J = 7.2 Hz, 1H), 7.55 (d, J = 10.8 Hz, 1H), 7.34 (s, 1H), 6.21 (s, 1H), 4.41 (s, 2H), 3.63 (t, J = 6.1 Hz, 2H), 2.79 (t, J = 6.1 Hz, 2H). |
| | N-(5-Chloro-2-fluoro-4-(6- | | |
| | fluorobenzo[c][1,2,5]oxadiazol-5- | | |
| | yl)phenyl)-6-oxo-3,4,6,7- | | |
| | tetrahydro-2,7-naphthyridine- | | |
| | 2(1H)-carboxamide | | |
| 9^{(d)} | 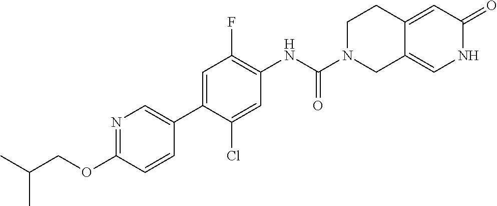 [SMILES: CC(C)COc1ccc(-c2cc(F)c(NC(=O)N3CCc4cc(=O)[nH]cc4C3)cc2Cl)cn1] N-(5-Chloro-2-fluoro-4-(6- | Method 1 471.4, 473.4 at 1.54 min | ¹H NMR (400 MHz, DMSO- d6) δ 11.40 (s, 1H), 8.46 (s, 1H), 8.21 (dd, J = 2.5, 0.7 Hz, 1H), 7.81 (dd, J = 8.6, 2.5 Hz, 1H), 7.75 (d, J = 7.4 Hz, 1H), 7.38 (d, J = 11.1 Hz, 1H), 7.33 (s, 1H), 6.90 (dd, J = 8.5, 0.7 Hz, 1H), 6.21 (s, 1H), 4.40 (s, 2H), 4.08 (d, J = 6.7 Hz, 2H), 3.61 (t, J = 6.1 Hz, 2H), 2.78 (t, J = 6.1 Hz, 2H), 2.05 (dp, J = 13.4, 6.7 Hz, 1H), 0.98 (d, J = 6.7 Hz, 6H). |
| | isobutoxypyridin-3-yl)phenyl)-6- | | |
| | oxo-3,4,6,7-tetrahydro-2,7- | | |
| | naphthyridine-2(1H)-carboxamide | | |
| 10^{(c)} | 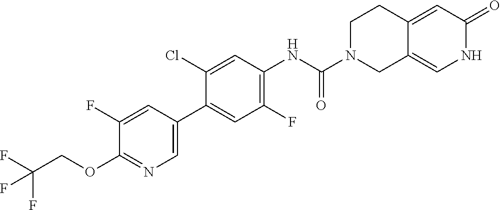 [SMILES: O=C(Nc1cc(Cl)c(-c2cnc(OCC(F)(F)F)c(F)c2)cc1F)N1CCc2cc(=O)[nH]cc2C1] | Method 1 515.3, 517.1 at 1.47 min | ¹H NMR (400 MHz, DMSO- d6) δ 11.42 (s, 1H), 8.51 (s, 1H), 8.13 (d, J = 2.0 Hz, 1H), 8.03 (dd, J = 11.2, 2.0 Hz, 1H), 7.78 (d, J = 7.3 Hz, 1H), 7.46 (d, J = 11.1 Hz, 1H), 7.34 (s, 1H), 6.21 (s, 1H), 5.15 (q, J = 9.0 Hz, 2H), 4.40 (s, 2H), 3.61 (t, J = 6.1 Hz, 2H), 2.78 (t, J = 6.1 Hz, 2H). |
| | N-(5-Chloro-2-fluoro-4-(5-fluoro-6- | | |
| | (2,2,2-trifluoroethoxy)pyridin-3- | | |
| | yl)phenyl)-6-oxo-3,4,6,7- | | |
| | tetrahydro-2,7-naphthyridine- | | |
| | 2(1H)-carboxamide | | |
| 11^{(c)} | 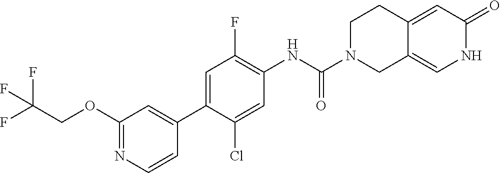 [SMILES: O=C(Nc1cc(Cl)c(-c2ccnc(OCC(F)(F)F)c2)cc1F)N1CCc2cc(=O)[nH]cc2C1] N-(5-Chloro-2-fluoro-4-(2-(2,2,2- | Method 1 497.3, 499.2 at 1.43 min | ¹H NMR (400 MHz, DMSO- d6) δ 8.58 (s, 1H), 8.28 (dd, J = 5.3, 0.7 Hz, 1H), 7.80 (d, J = 7.2 Hz, 1H), 7.49 - 7.42 (m, 2H), 7.23 (dd, J = 5.3, 1.5 Hz, 1H), 7.08 (t, J = 1.1 Hz, 1H), 6.32 (s, 1H), 5.05 (q, J = 9.1 Hz, 2H), 4.43 (s, 2H), 3.63 (t, J = 6.1 Hz, 4H), 2.82 (t, J = 6.1 Hz, 2H). (1 exchangeable proton not observed) |
| | trifluoroethoxy)pyridin-4-yl)phenyl)- | | |
| | 6-oxo-3,4,6,7-tetrahydro-2,7- | | |
| | naphthyridine-2(1H)-carboxamide | | |
| 12^{(a)} | 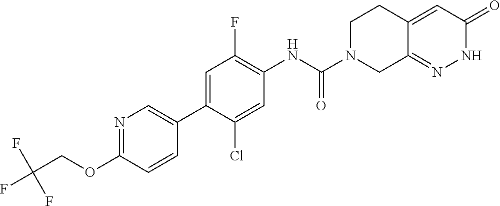 [SMILES: O=C(Nc1cc(Cl)c(-c2ccc(OCC(F)(F)F)nc2)cc1F)N1CCc2cc(=O)[nH]nc2C1] | Method 3 498.1 at 1.81 min | ¹H NMR (400 MHz, DMSO- d6) δ 12.88 (s, 1H), 8.71 (s, 1H), 8.28 (d, J = 2.0 Hz, 1H), 7.93 (dd, J = 2.4, 8.4 Hz, 1H), 7.75 (d, J = 7.2 Hz, 1H), 7.42 (d, J = 11.2 Hz, 1H), 7.09 (d, J = 8.4 Hz, 1H), 6.74 (s, 1H), 5.05 (q, J = 9.2 Hz, 2H), 4.53 (s, 2H), 3.68 (t, J = 6.0 Hz, 2H), 2.85 (t, J = 5.6 Hz, 2H). |
| | N-(5-Chloro-2-fluoro-4-(6-(2,2,2- | | |
| | trifluoroethoxy)pyridin-3-yl)phenyl)- | | |
| | 3-oxo-2,5,6,8-tetrahydropyrido[3,4- | | |
| | c]pyridazine-7(3H)-carboxamide | | |
| 13^{(a)} | 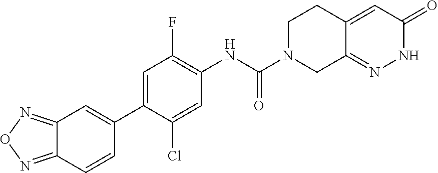 [SMILES: O=C(Nc1cc(Cl)c(-c2ccc3nonc3c2)cc1F)N1CCc2cc(=O)[nH]nc2C1] | Method 3 441.1 at 1.60 min | ¹HNMR (400 MHz, DMSO- d6) δ 12.89 (s, 1H), 8.78 (s, 1H), 8.16 - 8.12 (m, 2H), 7.82 (d, J = 7.2 Hz, 1H), 7.71 (d, J = 9.2 Hz, 1H), 7.55 (d, J = 10.8 Hz, 1H), 6.75 (s, 1H), 4.54 (s, 2H), 3.69 (t, J = 6.0 Hz, 2H), 2.86 (t, J = 5.6 Hz, 2H). |
| | N-(4-(Benzo[c][1,2,5]oxadiazol-5- | | |
| | yl)-5-chloro-2-fluorophenyl)-3-oxo- | | |
| | 2,5,6,8-tetrahydropyrido[3,4- | | |
| | c]pyridazine-7(3H)-carboxamide | | |
| 14^{(e)} | 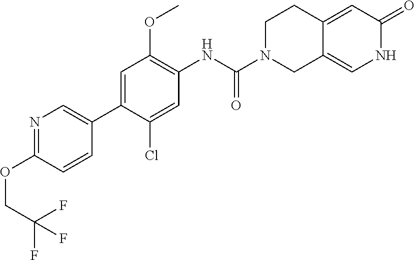 [SMILES: COc1cc(-c2ccc(OCC(F)(F)F)nc2)c(Cl)cc1NC(=O)N1CCc2cc(=O)[nH]cc2C1] | Method 5 509.0 at 1.56 min | ¹H NMR (500 MHz, DMSO- d6) δ 11.45 (s, 1H), 8.30 (d, J = 2.5 Hz, 1H), 7.99 (s, 1H), 7.94 (dd, J = 8.6, 2.5 Hz, 1H), 7.74 (s, 1H), 7.37 (s, 1H), 7.10 (t, J = 4.3 Hz, 2H), 6.22 (s, 1H), 5.06 (q, J = 9.1 Hz, 2H), 4.40 (s, 2H), 3.89 (s, 3H), 3.60 (t, J = 6.1 Hz, 2H), 2.79 (t, J = 6.2 Hz, 2H). |
| | N-(5-chloro-2-methoxy-4-(6-(2,2,2- | | |
| | trifluoroethoxy)pyridin-3-yl)phenyl)- | | |
| | 6-oxo-3,4,6,7-tetrahydro-2,7- | | |
| | naphthyridine-2(1H)-carboxamide | | |
| 15^{(e)} | 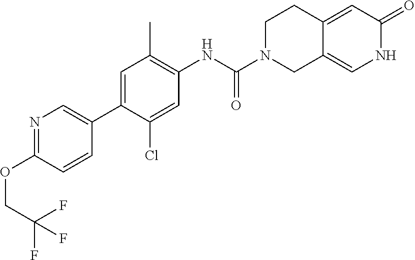 [SMILES: Cc1cc(-c2ccc(OCC(F)(F)F)nc2)c(Cl)cc1NC(=O)N1CCc2cc(=O)[nH]cc2C1] | Method 5 493.0 at 1.51 min | ¹H NMR (500 MHz, DMSO) δ 11.44 (s, 1H), 8.26 (d, J = 2.6 Hz, 1H), 8.12 (s, 1H), 7.91 (dd, J = 8.6, 2.5 Hz, 1H), 7.53 (s, 1H), 7.34 (s, 1H), 7.30 (s, 1H), 7.09 (d, J = 8.5 Hz, 1H), 6.22 (s, 1H), 5.06 (q, J = 9.1 Hz, 2H), 4.40 (s, 2H), 3.62 (t, J = 6.1 Hz, 2H), 2.80 (t, J = 6.2 Hz, 2H), 2.21 (s, 3H). |
| | N-(5-Chloro-2-methyl-4-(6-(2,2,2- | | |
| | trifluoroethoxy)pyridin-3-yl)phenyl)- | | |
| | 6-oxo-3,4,6,7-tetrahydro-2,7- | | |
| | naphthyridine-2(1H)-carboxamide | | |
| 16^{(f)} | 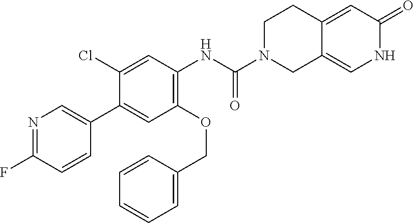 [SMILES: O=C(Nc1cc(Cl)c(-c2ccc(F)nc2)cc1OCc1ccccc1)N1CCc2cc(=O)[nH]cc2C1] | Method 1 505.4 at 1.34 min | ¹H NMR (500 MHz, DMSO) δ 11.46 (s, 1H), 8.31 (d, J = 2.6 Hz, 1H), 8.11 (td, J = 8.2, 2.6 Hz, 1H), 7.98 (s, 1H), 7.87 (s, 1H), 7.50 (d, J = 7.4 Hz, 2H), 7.39 (t, J = 7.4 Hz, 2H), 7.36 - 7.29 (m, 3H), 7.27 (s, 1H), 6.22 (s, 1H), 5.25 (s, 2H), 4.40 (s, 2H), 3.58 (t, J = 6.1 Hz, 2H), 2.77 (t, J = 6.1 Hz, 2H). |
| | N-(2-(Benzyloxy)-5-chloro-4-(6- | | |
| | fluoropyridin-3-yl)phenyl)-6-oxo- | | |
| | 3,4,6,7-tetrahydro-2,7- | | |
| | naphthyridine-2(1H)-carboxamide | | |

Key
(a) Product purified by Prep TLC;
(i) product isolated by filtration of reaction mixture;
(b) Product purified by chromatography on silica (0-15% MeOH/DCM);
(c) Product purified by chromatography on silica (0-10 % (0.7 M ammonia in MeOH/DCM);
(d) 2% NH₄OH in 3:1 EtOH/EtOAc) in isohexane;
(e) Product purified by chromatography on silica (0-100% 3:1EtOAc:EtOH/heptanes)
(f) Product purified by chromatography on silica (0-10% (1 Mammonia methanol/DCM)

### Intermediate 1 (I-1)

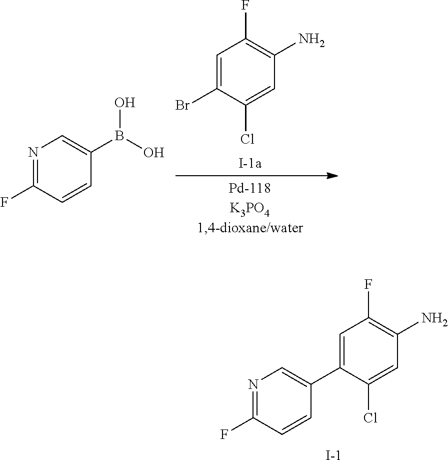 [SMILES: OB(O)c1ccc(F)nc1 ; Nc1cc(Cl)c(Br)cc1F ; Nc1cc(Cl)c(-c2ccc(F)nc2)cc1F ; [I-]]

To a vial was added 4-bromo-5-chloro-2-fluoroaniline I-1a (310 mg, 1.38 mmol), (6-fluoropyridin-3-yl)boronic acid (195 mg, 1.38 mmol) and Pd-118 (22.5 mg, 34.5 μmol). The vial was evacuated under vacuum and back filled with N₂. This was repeated 3 times. 1,4-dioxane (4 ml) was added, followed by an aqueous K₃PO₄ solution (2.07 ml, 2 M, 4.14 mmol), which had been sparged with N₂. After this addition, the vial was evacuated under vacuum and backfilled with N₂. The reaction was heated to 95° C. and stirred for 2 h. The reaction mixture was cooled to RT and filtered through a pad of Celite, washing with DCM (20 ml). The filtrate was diluted with water (4 ml) and the layers were separated. The aqueous phase was extracted with DCM (3×10 ml). The combined organics were dried over MgSO₄, filtered and concentrated in vacuo. The product was purified by chromatography on silica gel (0-30% EtOAc/isohexane) to afford 5-chloro-2-fluoro-4-(6-fluoropyridin-3-yl)aniline I-1 as an off-white powder. ¹H NMR (400 MHz, DMSO-d6) δ 8.23 (d, J=2.6 Hz, 1H), 8.02 (td, J=8.2, 2.6 Hz, 1H), 7.23 (dd, J=8.5, 2.9 Hz, 1H), 7.20 (d, J=11.9 Hz, 1H), 6.93 (d, J=8.3 Hz, 1H), 5.68 (s, 2H).

### Intermediate 2 (I-2)

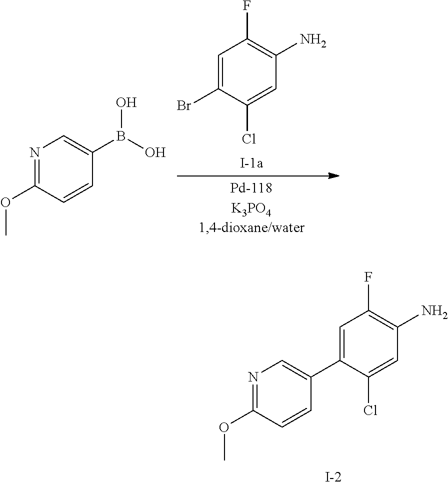

5-Chloro-2-fluoro-4-(6-methoxypyridin-3-yl)aniline (I-2) was synthesised from 4-bromo-5-chloro-2-fluoroaniline (I-1a) and 2-methoxy-5-pyridinylboronic acid using a procedure essentially the same as for I-1. ¹H NMR (400 MHz, DMSO-d6) δ 8.15 (dd, J=2.6, 0.7 Hz, 1H), 7.73 (dd, J=8.6, 2.5 Hz, 1H), 7.12 (d, J=11.9 Hz, 1H), 6.91 (d, J=8.3 Hz, 1H), 6.85 (dd, J=8.6, 0.8 Hz, 1H), 5.58 (s, 2H), 3.88 (s, 3H).

### Intermediate 3 (I-3)

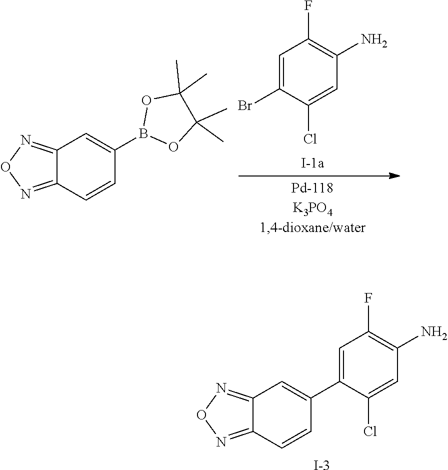

4-(Benzo[c][1,2,5]oxadiazol-5-yl)-5-chloro-2-fluoroaniline (I-3) was synthesised from 4-bromo-5-chloro-2-fluoroaniline (I-1a) and 5-(4,4,5,5-tetramethyl-1,3,2-dioxaborolan-2-yl)benzo[c][1,2,5]oxadiazole using a procedure essentially the same as for I-1. ¹H NMR (400 MHz, DMSO-d6) δ 8.06 (dd, J=9.4, 1.0 Hz, 1H), 8.00 (t, J=1.3 Hz, 1H), 7.68 (dd, J=9.3, 1.4 Hz, 1H), 7.31 (d, J=11.9 Hz, 1H), 6.95 (d, J=8.2 Hz, 1H), 5.81 (s, 2H).

### Intermediate 4 (I-4)

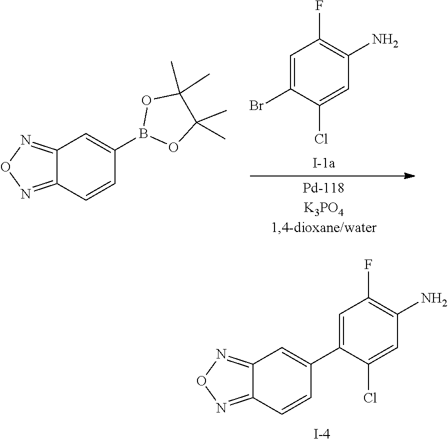

4-([1,2,5]oxadiazolo[3,4-b]pyridin-6-yl)-5-chloro-2-fluoroaniline (I-4) was synthesised from 4-bromo-5-chloro-2-fluoroaniline (I-1a) and 6-(4,4,5,5-tetramethyl-1,3,2-dioxaborolan-2-yl)-[1,2,5]oxadiazolo[3,4-b]pyridine using a procedure essentially the same as for I-1. ¹H NMR (400 MHz, DMSO-d6) δ 9.15 (d, J=2.1 Hz, 1H), 8.55 (d, J=2.1 Hz, 1H), 7.42 (d, J=11.9 Hz, 1H), 6.98 (d, J=8.2 Hz, 1H), 5.95 (s, 2H).

### Intermediate 5 (I-5)

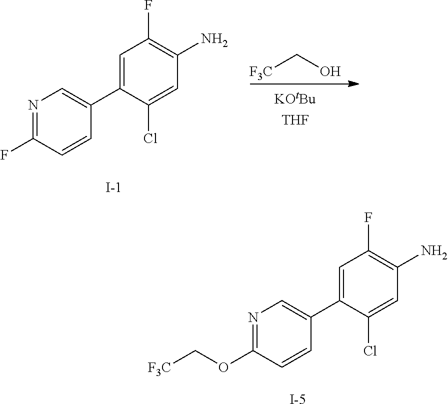

To a solution of 5-chloro-2-fluoro-4-(6-fluoropyridin-3-yl)aniline I-1 (10.00 g, 39.48 mmol) and trifluoroethanol (11.5 ml, 157.9 mmol) in THF (80 ml) at 0° C. was added a solution of potassium tert-butoxide in THF (95.4 ml, 20% w/w, 157.9 mmol). The reaction was allowed to warm to RT and stirred for 24 hours. Water (200 ml) was added and the layers were separated. The organic was washed with more water (200 ml) and separated. The aqueous phase was extracted with additional DCM (3×200 ml). The combined organics were dried over sodium sulfate and concentrated in vacuo. The product was purified by chromatography on silica gel (0-30% EtOAc/isohexane) to afford 5-chloro-2-fluoro-4-(6-(2,2,2-trifluoroethoxy)pyridin-3-yl)aniline I-5 as an orange oil. ¹H NMR (400 MHz, DMSO-d6) δ 8.19 (dd, J=2.6, 0.7 Hz, 1H), 7.84 (dd, J=8.6, 2.5 Hz, 1H), 7.15 (d, J=11.9 Hz, 1H), 7.03 (dd, J=8.5, 0.8 Hz, 1H), 6.92 (d, J=8.3 Hz, 1H), 5.63 (s, 2H), 5.02 (q, J=9.1 Hz, 2H).

### Intermediate 6 (I-6)

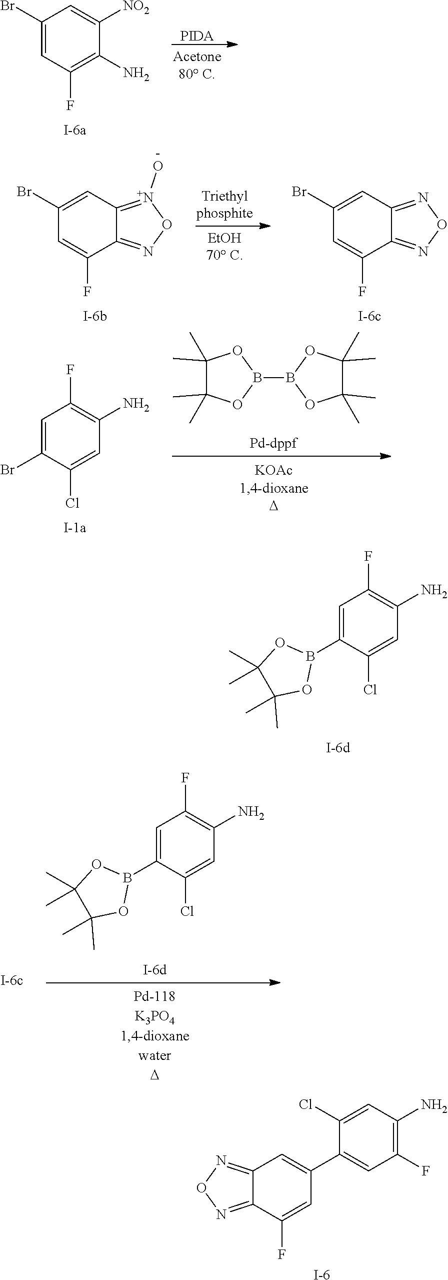

Step 1: To a solution of 4-bromo-2-fluoro-6-nitroaniline I-6a (500 mg, 2.13 mmol) in acetone (30 ml) was added PIDA (1.71 g, 5.32 mmol). The reaction mixture was heated to 80° C. for 6 h. The reaction was concentrated in vacuo to give a yellow solid. The product was purified by chromatography on silica gel (0-20% EtOAc/isohexane) to afford 6-bromo-4-fluorobenzo[c][1,2,5]oxadiazole 1-oxide I-6b as a pale yellow solid. ¹H NMR (400 MHz, DMSO-d6) δ 7.96 (s, 1H), 7.83-7.57 (m, 1H).

Step 2: To a solution of 5-bromo-7-fluorobenzo[c][1,2,5]oxadiazole 1-oxide I-6b (499 mg, 2.08 mmol) in EtOH (1.50 ml) was added triethyl phosphite (535 μl, 3.12 mmol) and the reaction mixture was heated to 70° C. for 2 h. The reaction was cooled to RT, diluted with DCM (15 ml) and 10% v/v aqueous sodium hypochlorite, then vigorously stirred for 20 min after which the dark brown solution passed through a phase separator and the filtrate was concentrated in vacuo. The product was purified by chromatography on silica gel (0-40% EtOAc/isohexane) to afford 6-bromo-4-fluorobenzo[c][1,2,5]oxadiazole I-6c as an orange oil that solidified over time to give an colourless oil.

Step 3: To a solution of 4-bromo-5-chloro-2-fluoroaniline I-1a (10.0 g, 44.6 mmol), potassium acetate (13.1 g, 134 mmol) and 4,4,4′,4′,5,5,5′,5′-octamethyl-2,2′-bi(1,3,2-dioxaborolane) (17.0 g, 66.8 mmol) was added 1,4-dioxane (100 ml) followed by Pd-dppf (2.73 g, 3.34 mmol). The reaction was stirred at reflux for 4 h before cooling to RT. The reaction mixture was diluted with EtOAc (200 ml) before being filtered through celite and the filter was washed with additional EtOAc (200 ml). The filtrates were combined and concentrated in vacuo. The product was partitioned between DCM (150 ml) and saturated NaHCO₃ (150 ml). The layers were separated and the organic phase was washed with additional saturated NaHCO₃ (150 ml) and brine (150 ml). The organics were concentrated in vacuo and the resulting residue was suspended in pentane (300 ml), stirred for 45 min and then cooled to 0° C. before being filtered. The precipitate was washed with additional pentane (2×50 ml) and dried in vacuo to afford 5-chloro-2-fluoro-4-(4,4,5,5-tetramethyl-1,3,2-dioxaborolan-2-yl)aniline I-6d as a black powder. ¹H NMR (400 MHz, DMSO-d6) δ 7.14 (d, J=11.9 Hz, 1H), 6.73 (d, J=7.7 Hz, 1H), 5.89 (s, 2H), 1.16 (s, 12H).

Step 4: 6-Bromo-4-fluorobenzo[c][1,2,5]oxadiazole I-6 was synthesised from 5-chloro-2-fluoro-4-(4,4,5,5-tetramethyl-1,3,2-dioxaborolan-2-yl)aniline I-6d and 6-bromo-4-fluorobenzo[c][1,2,5]oxadiazole I-6c using a procedure essentially the same as for I-1. ¹H NMR (400 MHz, DMSO-d6) δ 7.92 (d, J=1.0 Hz, 1H), 7.63 (dd, J=11.5, 1.1 Hz, 1H), 7.33 (d, J=12.0 Hz, 1H), 6.94 (d, J=8.3 Hz, 1H), 5.87 (s, 2H).

### Intermediate 7 (I-7)

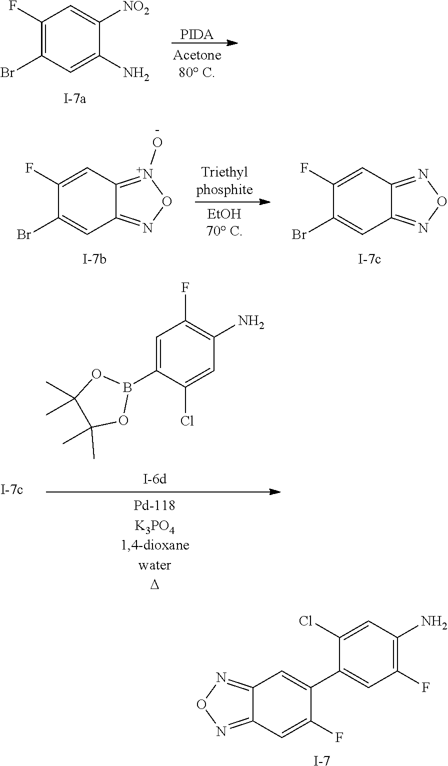

Step 1: 5-Bromo-2-fluoro-4-(6-fluorobenzo[c][1,2,5]oxadiazol-5-yl)aniline I-7b was synthesised from 4-bromo-2-fluoro-6-nitroaniline I-7a and PIDA using a procedure essentially the same as for I-6b. ¹H NMR (400 MHz, DMSO-d6) δ 8.39 (s, 1H), 7.93 (s, 1H).

Step 2: 5-Bromo-2-fluoro-4-(6-fluorobenzo[c][1,2,5]oxadiazol-5-yl)aniline I-7c was synthesised from triethyl phosphite and 5-bromo-2-fluoro-4-(6-fluorobenzo[c][1,2,5]-oxadiazol-5-yl)aniline I-7b using a procedure essentially the same as for I-6c. 1H NMR (400 MHz, DMSO-d6) δ 8.80 (dd, J=6.4, 0.6 Hz, 1H), 8.19 (dd, J=8.1, 0.6 Hz, 1H).

Step 3: 5-Chloro-2-fluoro-4-(6-fluorobenzo[c][1,2,5]oxadiazol-5-yl)aniline I-7 was synthesised from 5-chloro-2-fluoro-4-(4,4,5,5-tetramethyl-1,3,2-dioxaborolan-2-yl)aniline I-6d and 6-bromo-4-fluorobenzo[c][1,2,5]oxadiazole I-7c using a procedure essentially the same as for I-1. ¹H NMR (400 MHz, DMSO-d6) δ 8.17 (dd, J=6.7, 0.7 Hz, 1H), 8.05 (dd, J=9.2, 0.7 Hz, 1H), 7.27 (d, J=11.6 Hz, 1H), 6.95 (d, J=8.2 Hz, 1H), 5.83 (s, 2H).

### Intermediate 8 (I-8)

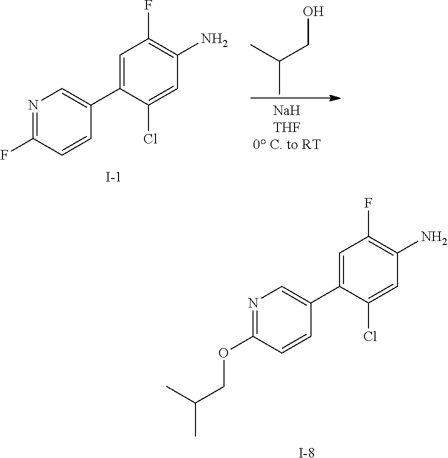

To a mixture of sodium hydride (96 mg, 60% w/w, 2.39 mmol) in THF (8 ml) at 0° C. was added 2-methylpropan-1-ol (210 μl, 2.28 mmol) and the resulting suspension was stirred at 0° C. for 45 min before 5-chloro-2-fluoro-4-(6-fluoropyridin-3-yl)aniline I-1 (360 mg, 1.50 mmol) was added. The resultant mixture was warmed to RT for 18 h. Water (0.1 ml) was added. The reaction mixture was partitioned between brine (25 ml) and DCM (25 ml) an the layers were separated. The aqueous was further extracted with DCM (25 ml) and the combined organics were concentrated in vacuo. The product was purified by chromatography on silica gel (0-10% (0.7 M Ammonia/MeOH)/DCM) to afford 5-chloro-2-fluoro-4-(6-isobutoxypyridin-3-yl)aniline I-8 as a tan solid. ¹H NMR (400 MHz, DMSO-d6) δ 8.12 (dd, J=2.5, 0.7 Hz, 1H), 7.72 (dd, J=8.6, 2.6 Hz, 1H), 7.11 (d, J=11.9 Hz, 1H), 6.90 (d, J=8.4 Hz, 1H), 6.84 (dd, J=8.6, 0.8 Hz, 1H), 5.59 (s, 2H), 4.05 (d, J=6.6 Hz, 2H), 2.03 (dq, J=13.4, 6.7 Hz, 1H), 0.97 (d, J=6.7 Hz, 6H).

### Intermediate 9 (I-9)

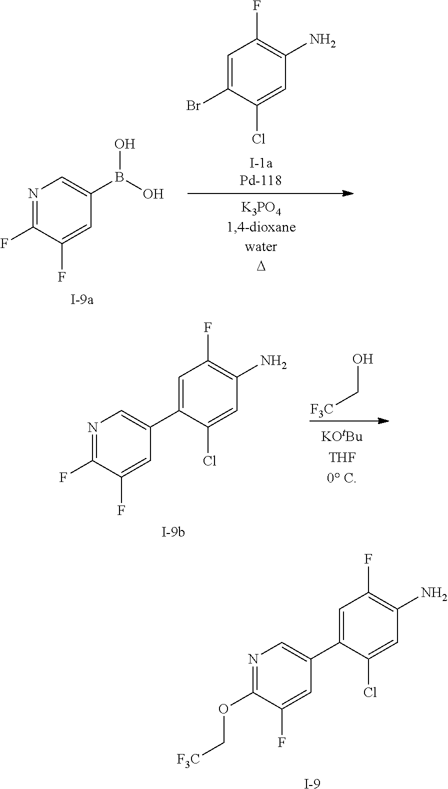

Step 1: 5-Chloro-4-(5,6-difluoropyridin-3-yl)-2-fluoroaniline (I-9b) was synthesised from 4-bromo-5-chloro-2-fluoroaniline (I-1a) and (5,6-difluoropyridin-3-yl)boronic acid I-9a using a procedure essentially the same as for I-1. ¹H NMR (400 MHz, DMSO-d6) δ 8.14 (ddd, J=10.8, 9.3, 2.1 Hz, 1H), 8.08 (t, J=1.9 Hz, 1H), 7.24 (d, J=11.9 Hz, 1H), 6.93 (d, J=8.3 Hz 1H), 5.76 (s, 2H).

Step 2: 5-Chloro-2-fluoro-4-(5-fluoro-6-(2,2,2-trifluoroethoxy)pyridin-3-yl)aniline (I-9) was synthesised from 5-chloro-4-(5,6-difluoropyridin-3-yl)-2-fluoroaniline (I-9b) and using a procedure essentially the same as for I-5. ¹H NMR (400 MHz, DMSO-d6) δ 8.04 (d, J=1.9 Hz, 1H), 7.89 (dd, J=11.3, 2.0 Hz, 1H), 7.20 (d, J=11.9 Hz, 1H), 6.92 (d, J=8.3 Hz, 1H), 5.69 (s, 2H), 5.12 (q, J=9.0 Hz, 2H).

### Intermediate 10 (I-10)

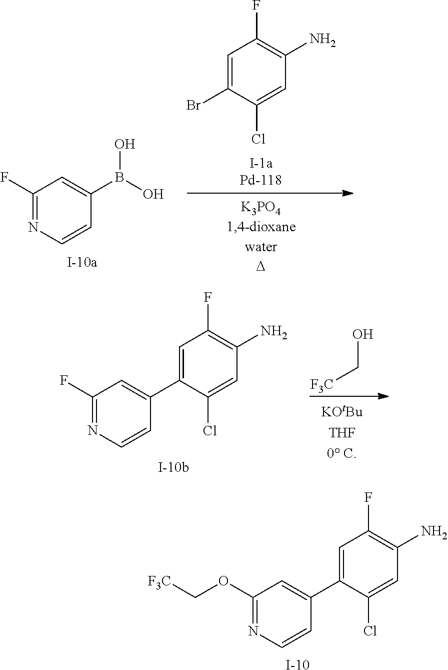

Step 1: 5-Chloro-2-fluoro-4-(2-fluoropyridin-4-yl)aniline (I-10b) was synthesised from 4-bromo-5-chloro-2-fluoroaniline (I-1a) and (2-fluoropyridin-4-yl)boronic acid I-10a using a procedure essentially the same as for I-1. ¹H NMR (400 MHz, DMSO-d6) δ 8.25 (d, J=5.2 Hz, 1H), 7.42 (ddd, J=5.3, 2.1, 1.4 Hz, 1H), 7.27 (d, J=12.0 Hz, 1H), 7.24-7.21 (m, 1H), 6.92 (d, J=8.2 Hz, 1H), 5.86 (s, 2H).

Step 2: 5-Chloro-2-fluoro-4-(2-(2,2,2-trifluoroethoxy)pyridin-4-yl)aniline (I-10) was synthesised from 5-chloro-2-fluoro-4-(2-fluoropyridin-4-yl)aniline (I-10b) and trifluoroethanol using a procedure essentially the same as for I-5. ¹H NMR (400 MHz, DMSO-d6) δ 8.21 (dd, J=5.3, 0.7 Hz, 1H), 7.21 (d, J=12.0 Hz, 1H), 7.17 (dd, J=5.3, 1.5 Hz, 1H), 6.98 (dd, J=1.5, 0.7 Hz, 1H), 6.91 (d, J=8.3 Hz, 1H), 5.78 (s, 2H), 5.02 (q, J=9.1 Hz, 2H).

### Intermediate 11 (I-11)

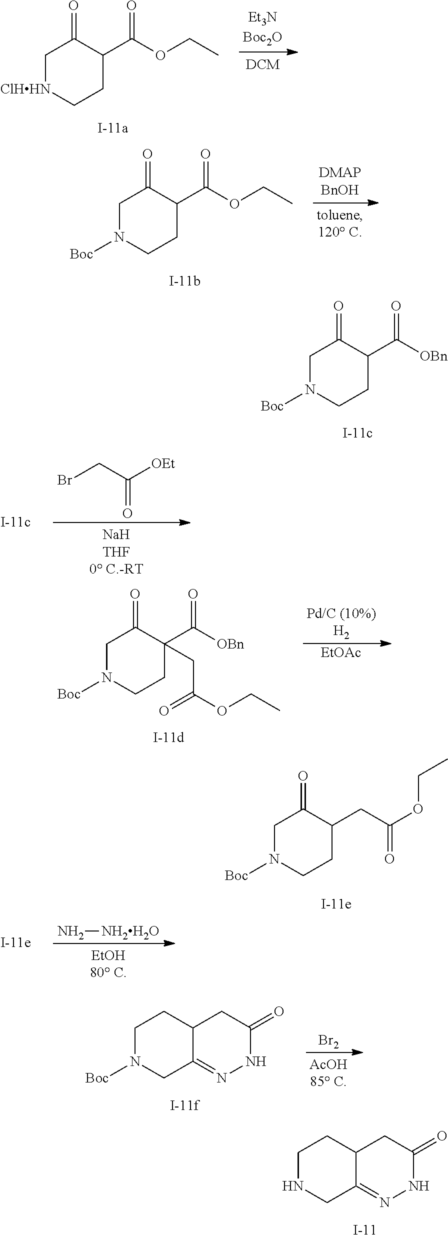

Step 1: To a solution of ethyl 3-oxopiperidine-4-carboxylate I-11a (2.0 g, 9.66 mmol) and Et₃N (2.62 ml, 19.3 mmol) in DCM (20 ml) was added di-tert-butyl dicarbonate (5.2 g, 24.15 mmol). The reaction mixture was stirred at RT for 16 h. The reaction mixture was diluted with water (20 ml) and the product was extracted with EtOAc (2×10 ml). The combined organics were dried over Na₂SO₄ and concentrated in vacuo. The product was purified by chromatography on silica gel (20% EtOAc/petroleum ether) to give 1-(tert-butyl) 4-ethyl 3-oxopiperidine-1,4-dicarboxylate I-11b as a yellow oil. ¹H NMR (400 MHz, DMSO-d6) δ 11.92 (s, 1H), 4.22-4.17 (m, 2H), 3.96 (s, 2H), 3.42-3.40 (m, 2H), 2.23-2.20 (m, 2H), 1.40 (s, 9H), 1.25-1.21 (m, 3H).

Step 2: To a solution of 1-(tert-butyl) 4-ethyl 3-oxopiperidine-1,4-dicarboxylate I-11b (2.3 g, 8.47 mmol) in toluene (40 ml) and BnOH (4.6 ml) was added DMAP (0.1 g, 0.84 mmol). The reaction mixture was stirred at RT for 16 h. The reaction mixture was diluted with water (30 ml) and the product was extracted with DCM (2×30 ml). The combined organics were dried over Na₂SO₄ and concentrated in vacuo. The product was purified by chromatography on silica gel (20% EtOAc/petroleum ether) to give 4-benzyl 1-(tert-butyl) 3-oxopiperidine-1,4-dicarboxylate I-11c as a yellow oil. ¹H NMR (400 MHz, DMSO-d6) δ 11.80 (s, 1H), 7.39-7.32 (m, 5H), 5.23 (s, 2H), 3.97 (s, 2H), 3.43-3.40 (m, 2H), 2.27-2.25 (m, 2H), 1.40 (s, 9H).

Step 3: To a solution of 4-benzyl 1-(tert-butyl) 3-oxopiperidine-1,4-dicarboxylate I-11c (1 g, 3.00 mmol) in THF (10 ml) was slowly added NaH (240 mg, 6.00 mmol) at 0° C. under N₂. The reaction mixture was stirred at RT for 1 h before ethyl 2-bromoacetate (0.66 ml, 6.00 mmol) was added. The reaction mixture was stirred at RT for another 16 h. The reaction was quenched with saturated aqueous NH₄Cl solution (10 ml) and the product was extracted with EtOAc (2×10 ml). The combined organics were washed with brine (10 ml), dried over Na₂SO₄ and concentrated. The product was purified by chromatography on silica gel (205 EtOAc/petroleum ether) to give 4-benzyl 1-(tert-butyl)4-(2-ethoxy-2-oxoethyl)-3-oxopiperidine-1,4-dicarboxylate I-11d as a yellow oil. ¹H NMR (400 MHz, DMSO-d6) δ 7.42-7.33 (m, 5H), 5.19-5.11 (m, 2H), 4.16-3.99 (m, 4H), 3.64-3.58 (m, 1H), 3.46-3.38 (m, 1H), 2.87-2.71 (m, 2H), 2.38-2.32 (m, 1H), 2.20-2.13 (m, 1H), 1.39 (s, 9H), 1.15 (t, J=7.2 Hz, 3H).

Step 4: To a solution of 4-benzyl 1-(tert-butyl) 4-(2-ethoxy-2-oxoethyl)-3-oxopiperidine-1,4-dicarboxylate I-11d (200 mg, 0.48 mmol) in MeOH (5 ml) was added Pd/C (20 mg, 10% w/w). The reaction was stirred at RT for 16 h under an atmosphere of H₂. The reaction mixture was filtered and the filtrate was concentrated in vacuo. The product was purified by prep-TLC (33% EtOAc/petroleum ether) to give tert-butyl 4-(2-ethoxy-2-oxoethyl)-3-oxopiperidine-1-carboxylate I-11e as a yellow oil. ¹H NMR (400 MHz, DMSO-d6) δ 4.08-3.99 (m, 3H), 3.89-3.77 (m, 2H), 3.37-3.28 (m, 1H), 2.90-2.82 (m, 1H), 2.62-2.56 (m, 1H), 2.41-2.35 (m, 1H), 2.08-2.02 (m, 1H), 1.74-1.63 (m, 1H), 1.41 (s, 9H), 1.18 (t, J=7.2 Hz, 3H).

Step 5: To a solution of tert-butyl 4-(2-ethoxy-2-oxoethyl)-3-oxopiperidine-1-carboxylate I-11e (70 mg, 0.24 mmol) in EtOH (2 ml) was added hydrazine hydrate (0.01 ml, 0.24 mmol 80% w/w). The reaction mixture was stirred at 80° C. for 1 h. The mixture was diluted with DCM (5 ml) and H₂O (5 ml) and the two layers were separated. The organic layer was dried over Na₂SO₄ and concentrated in vacuo to afford tert-butyl 3-oxo-2,4,4a,5,6,8-hexahydropyrido[3,4-c]pyridazine-7(3H)-carboxylate I-11f as a yellow oil. ¹H NMR (400 MHz, DMSO-d6) δ 10.60 (s, 1H), 4.31 (d, J=16.0 Hz, 1H), 4.18-3.98 (m, 1H), 3.83-3.71 (m, 2H), 3.06-2.90 (m, 1H), 2.77-2.67 (m, 1H), 2.43-2.37 (m, 1H), 2.23-2.15 (m, 1H), 1.99-1.93 (m, 1H), 1.40 (s, 9H).

Step 6: To a solution of tert-butyl 3-oxo-2,4,4a,5,6,8-hexahydropyrido[3,4-c]pyridazine-7(3H)-carboxylate I-11f (50 mg, 0.04 mmol) in AcOH (3 ml) was added Br₂ (32 mg, 0.20 mmol, 1 eq). After stirring at 85° C. for 1 h, the solvent was removed in vacuo to afford 5,6,7,8-tetrahydropyrido[3,4-c]pyridazin-3(2H)-one) I-11 as a brown solid, which was used without any further purification. LCMS (Method 3) m/z 152.2 (M+H)⁺ (ES⁺), at 0.26 min.

### Intermediate 12 (I-12)

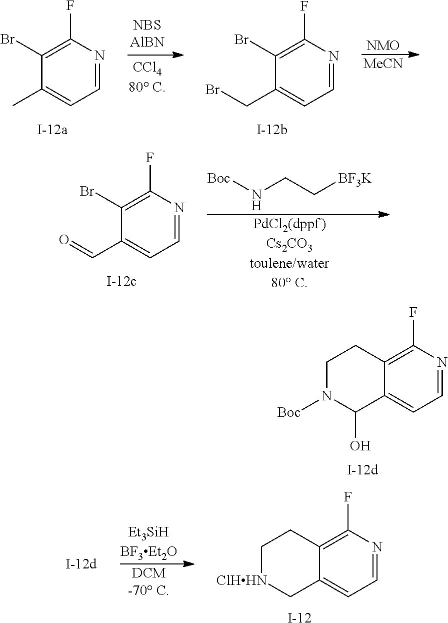

Step 1: To a mixture of 3-bromo-2-fluoro-4-methylpyridine I-12a (900 mg, 4.74 mmol) in CCl₄ (10 ml) were added NBS (1.01 g, 5.68 mmol) and AIBN (78 mg, 0.47 mmol). The mixture was stirred at 80° C. overnight. The reaction mixture was concentrated in vacuo and the product was purified by chromatography on silica gel (2% EtOAc/petroleum ether) to give 3-bromo-4-(bromomethyl)-2-fluoropyridine I-12b as a colourless oil. ¹H NMR (400 MHz, DMSO-d6) δ 8.23 (d, J=5.2 Hz, 1H), 7.60 (d, J=4.8 Hz, 1H), 4.75 (s, 2H).

Step 2: To a solution of 3-bromo-4-(bromomethyl)-2-fluoropyridine I-12b (540 mg, 2.01 mmol) in MeCN (10 ml) was added NMO (470 mg, 4.02 mmol). The resultant mixture was stirred at RT for 16 h. The reaction mixture was concentrated in vacuo and the product was purified by prep-TLC (20% EtOAc/petroleum ether) to give 3-bromo-2-fluoroisonicotinaldehyde I-12c as a white solid. ¹H NMR (400 MHz, DMSO-d6) δ 10.19 (s, 1H), 8.40 (d, J=4.8 Hz, 1H), 7.68 (dd, J=0.4 Hz, 4.8 Hz, 1H)

Step 3: To a solution of 3-bromo-2-fluoroisonicotinaldehyde I-12c (220 mg, 1.08 mmol) in toluene (6 ml) and H₂O (2 ml) were added potassium tert-butyl N-[2-(trifluoroboranuidyl)ethyl]carbamate (298 mg, 1.19 mmol), Cs₂CO₃ (1.05 g, 3.24 mmol) and PdCl₂(dppf) (39 mg, 52.92 μmol). The reaction mixture was stirred at 80° C. for 16 h. The reaction mixture was diluted with water (10 ml) and the product was extracted with EtOAc (3×5 ml). The combined organics were dried over Na₂SO₄, filtered and concentrated in vacuo. The product was purified by chromatography on silica gel (33% EtOAc/petroleum ether) to give tert-butyl 5-fluoro-1-hydroxy-3,4-dihydro-2,6-naphthyridine-2(1H)-carboxylate I-12d as a light yellow oil. ¹H NMR (400 MHz, DMSO-d6) δ 8.07 (d, J=4.8 Hz, 1H), 7.32 (d, J=5.2 Hz, 1H), 6.68 (s, 1H), 6.19 (s, 1H), 4.09 (s, 1H), 3.17 (s, 1H), 2.75-2.57 (m, 2H), 1.44 (s, 9H).

Step 4: To a mixture of tert-butyl 5-fluoro-1-hydroxy-3,4-dihydro-2,6-naphthyridine-2(1H)-carboxylate I-12d (40 mg, 0.15 mmol) in DCM (10 ml) were added triethylsilane (52 mg, 0.45 mmol) and boron trifluoride diethyl etherate (132 mg, 0.45 mmol, 48% w/w) dropwise at −70° C. The resultant mixture was stirred at −70° C. for 2 h. The reaction was quenchened with a saturated aqueous NaHCO₃ solution (10 ml) and the product was extracted with DCM (3×5 ml). The combined organics were dried over Na₂SO₄, filtered and concentrated in vacuo to give 5-fluoro-1,2,3,4-tetrahydro-2,6-naphthyridine hydrochloride I-12 as a brown oil, which was used directly without any further purification. LCMS (Method 3) m/z 153.0 (M+H)⁺ (ES⁺), at 0.39 min

### Intermediate 13 (I-13)

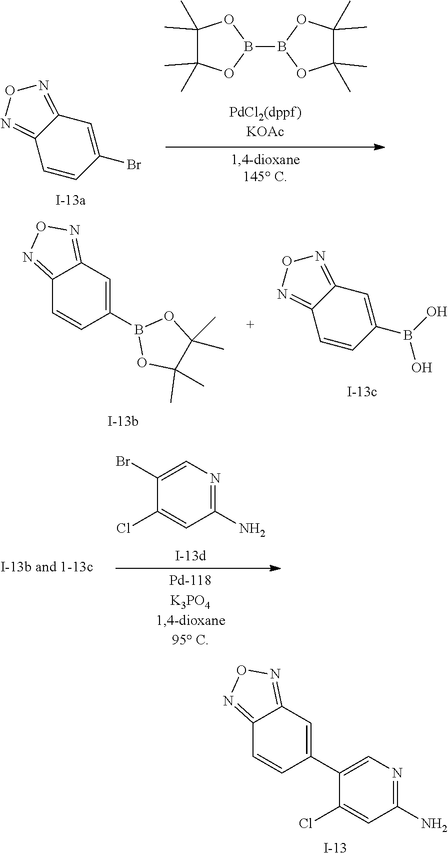

Step 1: To a mixture of 5-bromobenzo[c][1,2,5]oxadiazole I-13a (1 g, 5.02 mmol) and 4,4,4′,4′,5,5,5′,5′-octamethyl-2,2′-bi(1,3,2-dioxaborolane) (1.53 g, 6.02 mmol) in 1,4-dioxane (12 ml) were added PdCl₂(dppf) (365 mg, 0.50 mmol) and KOAc (1.48 g, 15.07 mmol). The reaction mixture was heated at 145° C. using microwave conditions for 30 min. The mixture was concentrated in vacuo and the mixture of 5-(4,4,5,5-tetramethyl-1,3,2-dioxaborolan-2-yl)benzo[c][1,2,5]oxadiazole I-13b and benzo[c][1,2,5]oxadiazol-5-ylboronic acid I-13c was used in the next step without any further purification.

Step 2: To a solution of 5-bromo-4-chloropyridin-2-amine (1 g, 4.82 mmol) and the mixture of 5-(4,4,5,5-tetramethyl-1,3,2-dioxaborolan-2-yl)benzo[c][1,2,5]oxadiazole I-13b and benzo[c][1,2,5]oxadiazol-5-ylboronic acid I-13c in 1,4-dioxane (10 ml) were added Pd-118 (79 mg, 0.12 mmol) and K₃PO₄ (7.2 ml, 2 M in water 14.46 mmol) at RT at 95° C. under N₂ atmosphere. The reaction mixture was diluted with water (10 ml) and the product was extracted with EtOAc (3×5 ml). The combined organics were dried over Na₂SO₄, filtered and concentrated in vacuo. The product was purified by chromatography on silica gel (2% THF in DCM) to give 5-(benzo[c][1,2,5]oxadiazol-5-yl)-4-chloropyridin-2-amine I-13 as a yellow solid. LCMS (Method 4) m/z 247.2 (M+H)⁺ (ES⁺), at 3.13 min. ¹H NMR (400 MHz, DMSO-d6) δ 8.09-8.06 (m, 2H), 8.03 (s, 1H), 7.68 (dd, J=1.2, 9.2 Hz, 1H), 6.96 (s, 1H), 6.58 (s, 2H).

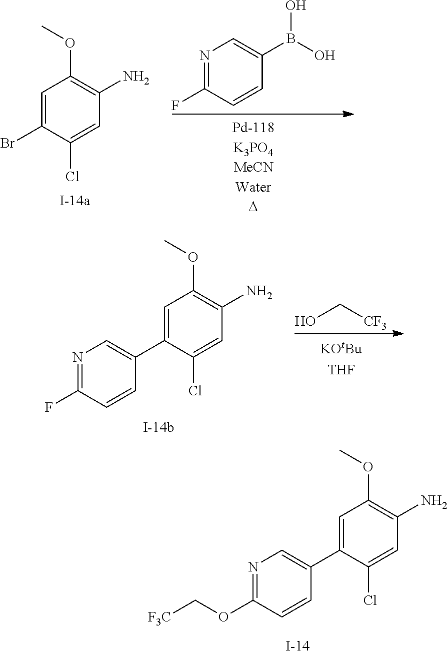

Step 1: To a solution of (6-fluoropyridin-3-yl)boronic acid (328 mg, 2.33 mmol), 4-bromo-5-chloro-2-methoxyaniline I-14a (0.500 g, 2.11 mmol) and 1,1′-Bis(di-t-butylphosphino)-ferrocene palladium dichloride (34.4 mg, 52.9 μmol) in MeCN (3.00 mL) was added a solution of potassium phosphate (2.11 mL, 2 M, 4.23 mmol). The mixture was evacuated under vacuum and purged with N₂ (3 times). The mixture was heated at 60° C. for 1 h, after which the reaction mixture was cooled, water (15 mL) and EtOAc (15 mL) were added. The layers were separated and the aqueous was extracted with EtOAc (2×20 mL). The combined organics were washed with brine (20 mL), dried with sodium sulfate and the mixture was concentrated in vacuo. The product was purified by chromatography on silica gel (0-100% EtOAc/isohexane) to afford a pink solid. EtOAc (5 ml) and heptane (30 ml) was added and the flask was swirled. The resultant precipitate was filtered and dried in vacuo to give 5-chloro-4-(6-fluoropyridin-3-yl)-2-methoxyaniline I-14b as a colourless solid. ¹H NMR (500 MHz, DMSO-d6) δ 8.26 (d, J=2.5 Hz, 1H), 8.03 (td, J=8.2, 2.6 Hz, 1H), 7.24 (dd, J=8.4, 2.8 Hz, 1H), 6.87 (s, 1H), 6.78 (s, 1H), 5.25 (s, 2H), 3.81 (s, 3H).

Step 2: 5-Chloro-2-methoxy-4-(6-(2,2,2-trifluoroethoxy)pyridin-3-yl)aniline I-14 was synthesised from 5-chloro-4-(6-fluoropyridin-3-yl)-2-methoxyaniline I-14b and trifluoroethanol using a procedure essentially the same as for I-5. ¹H NMR (500 MHz, DMSO-d6) δ 8.22 (d, J=2.4 Hz, 1H), 7.86 (dd, J=8.5, 2.5 Hz, 1H), 7.04 (d, J=8.5 Hz, 1H), 6.83 (s, 1H), 6.77 (s, 1H), 5.19 (s, 2H), 5.04 (q, J=9.1 Hz, 2H), 3.81 (s, 3H).

### Intermediate 15 (I-15)

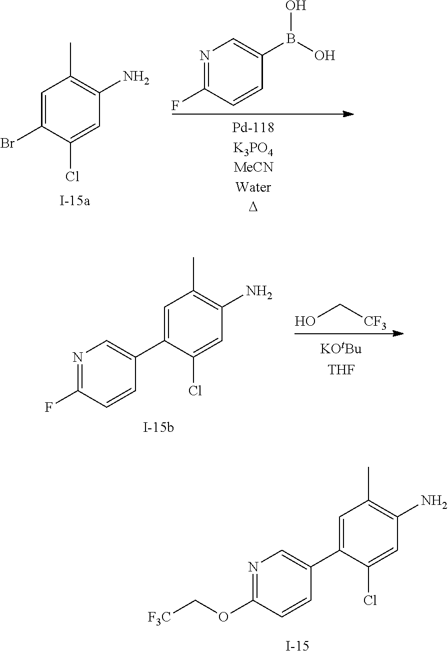

Step 1: 5-Chloro-4-(6-fluoropyridin-3-yl)-2-methylaniline I-15b was synthesised from 4-bromo-5-chloro-2-methylaniline I-15a and (6-fluoropyridin-3-yl)boronic acid using a procedure essentially the same as for I-14b. ¹H NMR (500 MHz, DMSO-d6) δ 8.20 (d, J=2.5 Hz, 1H), 7.98 (td, J=8.2, 2.6 Hz, 1H), 7.21 (dd, J=8.7, 2.8 Hz, 1H), 7.04 (d, J=0.9 Hz, 1H), 6.78 (s, 1H), 5.37 (s, 2H), 2.07 (s, 3H).

Step 2: 5-Chloro-2-methyl-4-(6-(2,2,2-trifluoroethoxy)pyridin-3-yl)aniline I-15 was synthesised from 5-chloro-4-(6-fluoropyridin-3-yl)-2-methylaniline I-15b and trifluoroethanol using a procedure essentially the same as for I-5. ¹H NMR (500 MHz, DMSO-d6) δ 8.16 (d, J=2.4 Hz, 1H), 7.80 (dd, J=8.5, 2.5 Hz, 1H), 7.05-6.99 (m, 2H), 6.77 (s, 1H), 5.31 (s, 2H), 5.02 (q, J=9.1 Hz, 2H), 2.07 (s, 3H).

### Intermediate 16 (I-16)

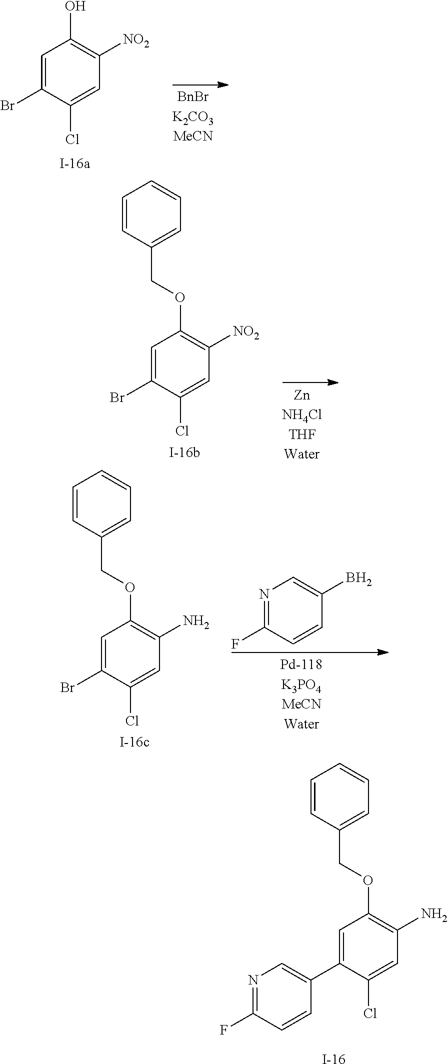

Step 1: A solution of benzyl bromide (1.04 mL, 8.71 mmol), 5-bromo-4-chloro-2-nitrophenol I-16a (2.00 g, 7.92 mmol) and potassium carbonate (3.28 g, 23.8 mmol) in MeCN (20 mL) was stirred at 60° C. for 6 h. The reaction mixture was concentrated in vacuo. Water (20 mL) and EtOAc (20 mL) were added and the layers were separated. The organics were washed with an aqueous sodium hydroxide solution (10 ml, 2 M) and brine (10 ml) and dried with magnesium sulfate. The organics were concentrated in vacuo to give 1-(benzyloxy)-5-bromo-4-chloro-2-nitrobenzene I-16b as light red solid. ¹H NMR (500 MHz, DMSO-d6) δ 8.26 (s, 1H), 7.95 (s, 1H), 7.48-7.33 (m, 5H), 5.36 (s, 2H).

Step 2: To a solution of 1-(benzyloxy)-5-bromo-4-chloro-2-nitrobenzene I-16b (2.67 g, 7.79 mmol) in THF (20 ml) and water (10 ml) was added ammonium chloride (2.50 g, 46.8 mmol) and zinc (3.06 g, 46.8 mmol) and the mixture was stirred at RT for 6 h. The reaction mixture was concentrated in vacuo and the residue was partitioned between ethyl acetate (30 ml) and water (10 ml). The organics were washed with water (10 ml), brine (10 ml) and dried with magnesium sulfate. The organics were concentrated in vacuo to give 2-(benzyloxy)-4-bromo-5-chloroaniline I-16c as a brown solid. ¹H NMR (500 MHz, DMSO-d6) δ 7.48 (d, J=7.5 Hz, 2H), 7.39 (t, J=7.6 Hz, 2H), 7.33 (d, J=7.3 Hz, 1H), 5.22 (s, 2H), 5.12 (s, 2H).

Step 3: 2-(Benzyloxy)-5-chloro-4-(6-fluoropyridin-3-yl)aniline I-16 was synthesised from 2-(benzyloxy)-4-bromo-5-chloroaniline I-16c and (6-fluoropyridin-3-yl)boronic acid using a procedure essentially the same as for I-14b. ¹H NMR (500 MHz, DMSO-d6) δ 8.21 (s, 1H), 8.00 (t, J=8.3 Hz, 1H), 7.49 (d, J=7.6 Hz, 2H), 7.39 (t, J=7.6 Hz, 2H), 7.36-7.29 (m, 1H), 7.27-7.19 (m, 1H), 6.99 (s, 1H), 6.81 (s, 1H), 5.28 (s, 2H), 5.15 (s, 2H).

Human GPR65 Cyclic Adenosine Monophosphate (cAMP) Homogeneous Time Resolved Fluorescence (HTRF) Antagonist Assay Procedure

IC₅₀ data was obtained by the following procedure:

1321N1 human astrocytoma cells stably expressing human recombinant GPR65 (1321N1-hrGPR65 cells, EuroscreenFast) were cultured according to the vendor's instructions.

Compounds were tested for their ability to antagonise GPR65, through measuring the concentration of cytoplasmic cAMP following treatment of the cells at a pH of 7.2 to activate GPR65 signalling and addition of the compound to be tested. The extent to which the expected rise in cAMP concentration upon GPR65 activation was suppressed by the added compound is indicative of its potency. The assay was carried out according to EuroscreenFast assay Methodology as follows.

On the day of the assay, test compounds were added to 384-well, low volume, white microtiter plates by acoustic dispensing. KRH buffer (5 mM KCl, 1.25 mM MgSO₄, 124 mM NaCl, 25 mM HEPES, 13.3 mM Glucose, 1.25 mM KH₂PO₄ and 1.45 mM CaCl₂) was adjusted to pH 6.5, pH 7.6 and pH 8.4 by adding NaOH. 1321N1-hGPR65 cells were rapidly thawed and diluted in KRH, pH 7.6 prior to centrifugation at 300×g for 5 min and resuspension in assay buffer (KRH, pH 7.6, supplemented with 1 mM 3-isobutyl-1-methylxanthine (IBMX) and 200 μM ethylenediaminetetraacetic acid (EDTA)). Cells were added to assay plates at a density of 2,000 cells per well in a volume of 5 μl. Assay plates were briefly centrifuged at 100×g and then incubated at room temperature for 30 min. Cells were stimulated by the addition of 5 μL KRH, pH 6.5, to achieve an assay pH of 7.2, while control wells received 5 μl KRH, pH 8.4 to achieve an assay pH of 7.9. Assay plates were briefly centrifuged at 100×g and then incubated at room temperature for 30 min.

Accumulation of cAMP was detected by cAMP HTRF kit (Cisbio). d2-labeled cAMP and cryptate-labeled anti-cAMP antibody in Lysis and Detection Buffer (Cisbio) were added to assay plates, and the plates were incubated at room temperature for 1 h. HTRF measurements were performed using a Pherastar FSX instrument. Acceptor and donor emission signals were measured at 665 nm and 620 nm, respectively, and HTRF ratios were calculated as signal_{665 nm}/signal_{620 nm}×10⁴. Data were normalised to high and low control values and fitted with 4-parameter logistic regression to determine hGPR65 IC50 values for the test compounds, which are shown in Table 1.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Activity of selected compounds according to the invention

| | |
|---|---|
| 1 | High |
| 2 | High |
| 3 | Medium |
| 4 | Medium |
| 5 | High |
| 6 | High |
| 7 | High |
| 8 | High |
| 9 | High |
| 10 | High |
| 11 | High |
| 12 | High |
| 13 | High |
| 14 | Medium |
| 15 | Medium |
| 16 | Low |

High = IC50 < 500 nM;
Medium = IC50 > 500 nM and <5 μM;
Low > 5 μM

### REFERENCES

Bohn, T. et al. (2018). Tumor immunoevasion via acidosis-dependent induction of regulatory tumor-associated macrophages. Nature Immunology, 1319-1326.
Damaghi, M. et al. (2013). pH Sensing and Regulation in Cancer. Frontiers in Physiology.
Fingl et al, 1975, The Pharmacological Basis of Therapeutics, chapter 1, page 1
Gaublomme, J. et al. (2015). Single-Cell Genomics Unveils Critical Regulators of Th17 Cell Pathogenicity. Cell, 1400-1412.
Hernandez, J. (2018). GPR65, a critical regulator of Th17 cell pathogenicity, is regulated by the CRTC2/CREB pathway. The Journal of Immunology, 200 (Supplement).
Korn, T. et al. (2009). IL-17 and Th17 Cells. Annual Reviews in Immunology, 485-517.
Wang, J. et al. (2004). TDAG8 is a proton-sensing and psychosine-sensitive G-protein-coupled receptor. Journal of Biological Chemistry, 45626-45633.
Yoshida, N. et al. (2016). ICER is requisite for Th17 differentiation. Nature Communications, 12993.
Hardin, M. et al. (2014). The clinical and genetic features of COPD-asthma overlap syndrome. Eur Respir J. 2014 August; 44(2):341-50.
Kottyan, L. et al. (2009). Eosinophil viability is increased by acidic pH in a cAMP- and GPR65-dependent manner. Blood. 2009 Sep. 24; 114(13):2774-82.
Tsurumaki, H. et al (2015). Int J Mol Sci. Protective Role of Proton-Sensing TDAG8 in Lipopolysaccharide-Induced Acute Lung Injury. December 4; 16(12):28931-42

1. A compound of formula (I), or a pharmaceutically acceptable salt or solvate thereof, 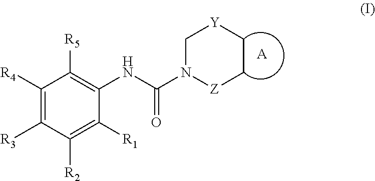
wherein:
ring A is a 5- or 6-membered monocyclic aromatic or heteroaromatic ring, or a 9- or 10-membered bicyclic aromatic or heteroaromatic ring, each of which is optionally substituted with one or more substituents independently selected from halo, CN, alkoxy, NR₁₁R₁₁′, OH, SO₂-alkyl, CO₂-alkyl, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R₁₁′, OH, alkyl, haloalkyl, and aralkyl;
Y and Z are each independently CR₁₀R₁₀′, wherein R₁₀ and R₁₀′ are each independently selected from H, F, alkyl, and haloalkyl;
R₁, R₄, and R₅ are each independently selected from H, halo, alkyl, alkoxy, O-aralkyl and haloalkyl;
at least one of R₂ and R₃ is a monocyclic or fused bicyclic aryl or a monocyclic or fused bicyclic heteroaryl group, each of which is optionally substituted by one or more substituents selected from alkyl, alkoxy, haloalkyl, haloalkoxy, halo, aryl, heteroaryl, CONR₁₄R₁₄′, (CH₂)ₘNR₁₁R₁₁′, SO₂-alkyl, CN, hydroxyalkyl, (CH₂)ₘ-heterocycloalkyl, (CH₂)ₘ-cycloalkyl, CO₂R₁₅, alkoxy-alkyl and O-cycloalkyl;
the other of R₂ and R₃ is selected from halo, CN, and haloalkyl;
each R₁₁ and each R₁₁′ is independently selected from H, alkyl, haloalkyl, COR₁₂, and SO₂R₁₃, wherein R₁₂ and R₁₃ are both alkyl;
R₁₄, R₁₄′ and R₁₅ are each independently selected from H, alkyl and alkoxyalkyl; and
each m is independently 0 to 3;
with the proviso that the compound is other than:
N-(3-fluoro-4-(4-methyl-1H-imidazol-1-yl)phenyl)-6-methoxy-3,4-dihydroisoquinoline-2(1H)-carboxamide;
N-(3-fluoro-4-(4-methyl-1H-imidazol-1-yl)phenyl)-3,4-dihydroisoquinoline-2(1H)-carboxamide;
N-(3-fluoro-4-(4-methyl-1H-imidazol-1-yl)phenyl)-6,7-dimethoxy-3,4-dihydroisoquinoline-2(1H)-carboxamide.

2. A compound according to claim 1, wherein R₁ is selected from H and F, and is preferably H.

3. A compound according to claim 1 or claim 2, wherein:
R₃ is a monocyclic or fused bicyclic aryl or a monocyclic or fused bicyclic heteroaryl group, each of which is optionally substituted by one or more substituents selected from alkyl, alkoxy, haloalkyl, haloalkoxy, halo, aryl, heteroaryl, CONR₁₄R₁₄′, (CH₂)ₘNR₁₁R₁₁′, SO₂-alkyl, CN, hydroxyalkyl, (CH₂)ₘ-heterocycloalkyl, (CH₂)ₘ-cycloalkyl, CO₂R₁₅, alkoxy-alkyl and O-cycloalkyl; and
R₂ is selected from halo, CN, and haloalkyl.

4. A compound according to any preceding claim, wherein R₂ is halo, more preferably, Cl.

5. A compound according to any preceding claim, wherein R₃ is selected from phenyl, pyridinyl, pyrimidinyl, pyridazinyl, pyrazinyl, isoxazolyl, oxazolyl, thiazolyl, isothiazolyl, thiadiazoyl, triazinyl, thienyl, furanyl, pyrrolyl, imidazolyl, pyrazolyl, oxadiazolyl, triazolyl, benzofuranyl, benzothienyl, pyrrolopyridinyl, benzoxazinyl, benzimidazolyl, benzotriazolyl, benzoxadiazolyl, benzothiadiazolyl, benzothiazolyl, oxadiazolopyridinyl, indazolinyl, imidazopyridinyl, benzoisoxazolyl, benzoxazolyl, indolyl, isoindolyl, oxoisoindolinyl, oxoindolinyl, indazolyl, quinolinyl, isoquinolinyl, cinnolinyl, phthalazinyl, quinazolinyl, quinoxalinyl and naphthyridinyl, each of which is optionally substituted by one or more substituents selected from alkyl, alkoxy, haloalkyl, haloalkoxy and halo.

6. A compound according to any preceding claim, wherein R₃ is selected from 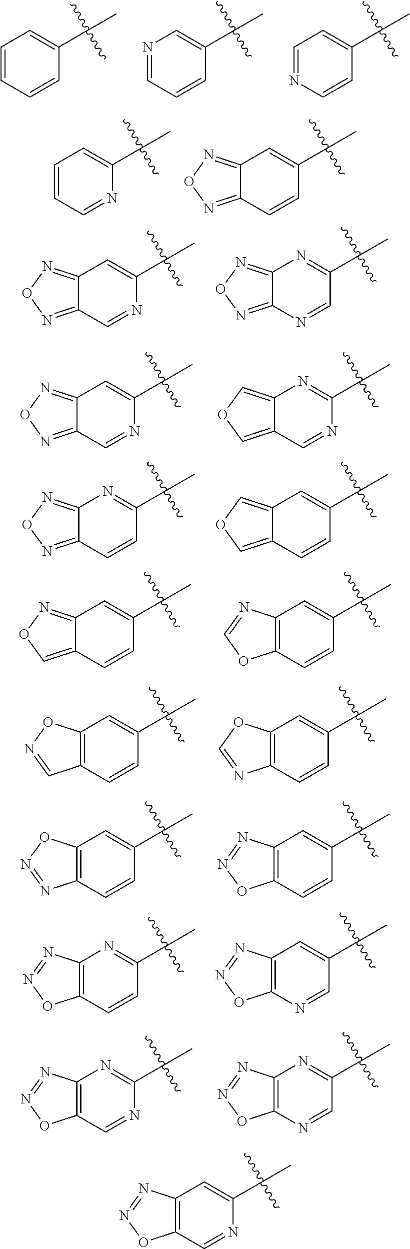
each of which is optionally substituted by one or more substituents selected from alkyl, halo, haloalkyl, alkoxy and haloalkoxy, preferably wherein R₃ is selected from 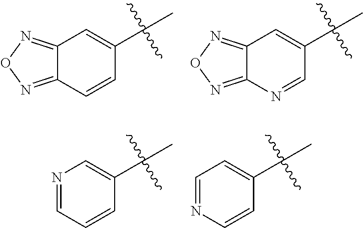 [SMILES: *c1ccc2nonc2c1 ; *c1cnc2nonc2c1 ; *c1cccnc1 ; *c1ccncc1]
each of which is optionally substituted by one or more substituents selected from F, alkoxy and fluoroalkoxy.

7. A compound according to any preceding claim, wherein R₃ is selected from

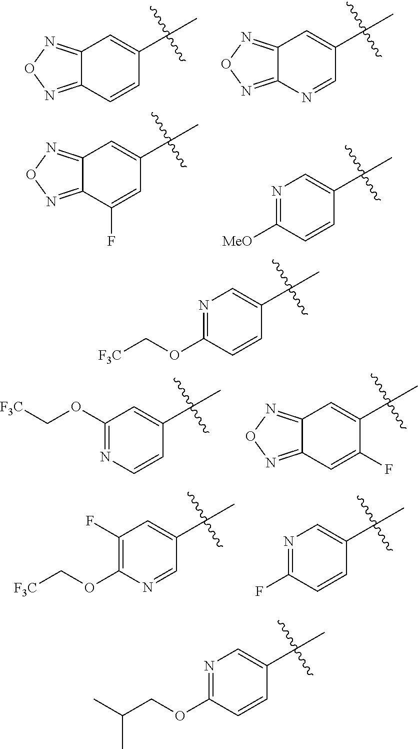

8. A compound according to any preceding claim, wherein R₄ is H or Cl, and is more preferably H.

9. A compound according to any preceding claim, wherein R₅ is H, alkyl, O-aralkyl or halo, preferably halo.

10. A compound according to any preceding claim, wherein R₅ is H, Me, O-benzyl or F, preferably F.

11. A compound according to any preceding claim, wherein the monocyclic aromatic or heteroaromatic ring A is a group selected from benzene, pyridine, pyridone, pyridine N-oxide, pyridazine, pyrimidine, pyrimidone, pyrazine, triazine, pyrrole, furan, thiophene, pyrazole, isoxazole, imidazole, oxazole, oxadiazole and thiazole, each of which may be optionally substituted.

12. A compound according to preceding claim, wherein ring A is a group selected from benzene, pyridine, pyridone, pyridine N-oxide, pyrimidine, pyrimidone, pyridazine, pyrazine, and isoxazole, each of which is optionally substituted with one or more substituents selected from F, Cl, Br, I, CN, C₁-C₆ alkoxy, NR₁₁R₁₁′, OH, C₁-C₆ alkyl, phenyl, SO₂—C₁-C₆alkyl, CO₂—C₁-C₆ alkyl, thienyl, halo-substituted pyridinyl, and C₁-C₆ haloalkyl.

13. A compound according to any preceding claim, wherein ring A is selected from: 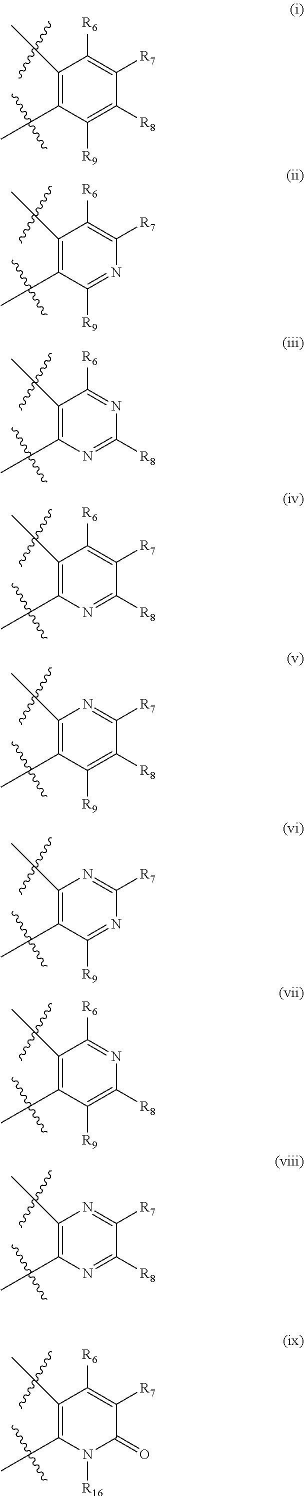 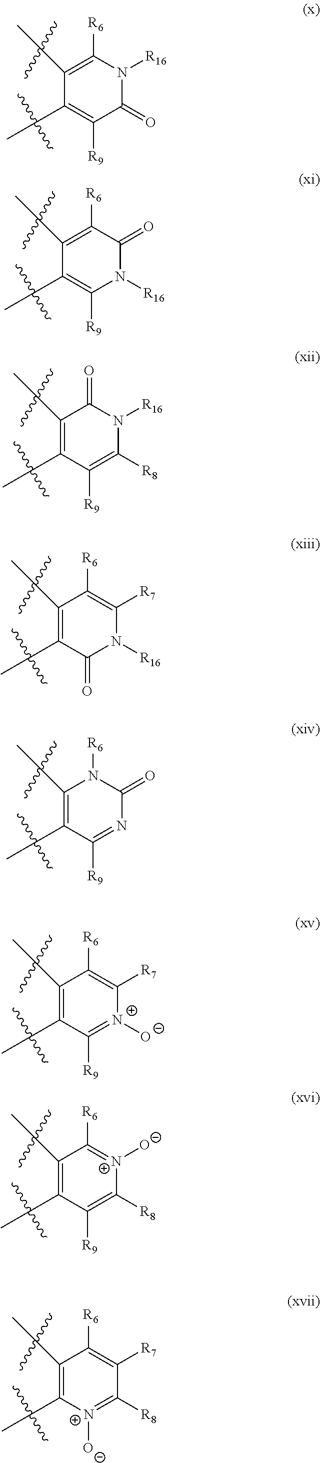 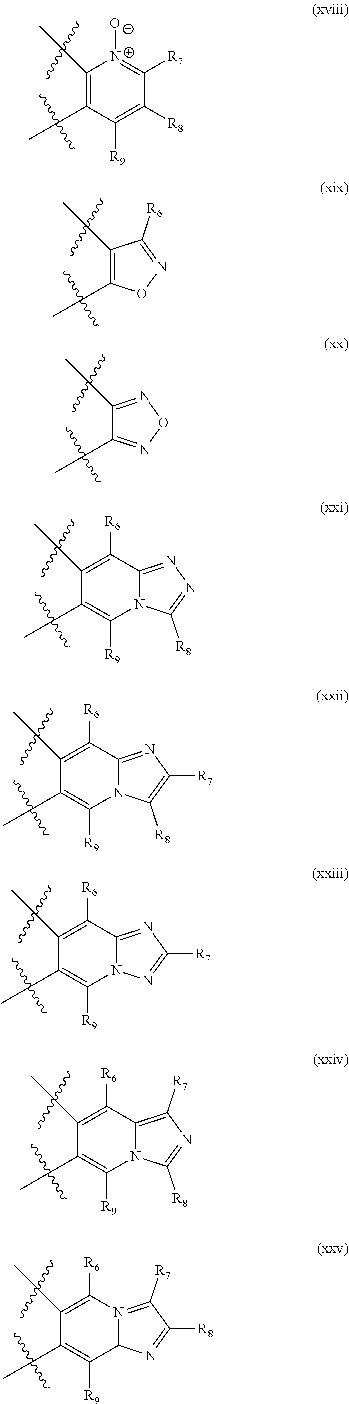 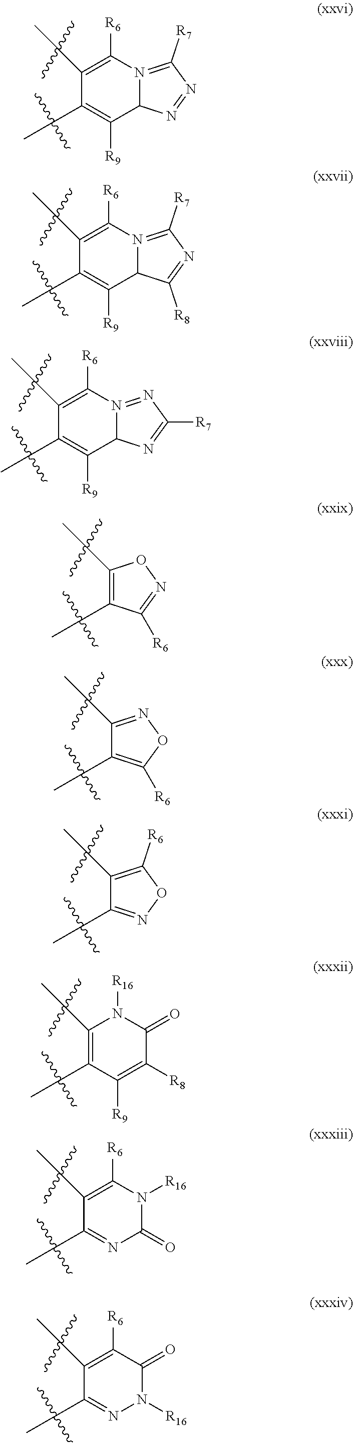
wherein R₆, R₇, R₈, and R₉ are each independently selected from H, halo, CN, alkoxy, CO₂-alkyl, SO₂-alkyl, NR₁₁R₁₁′, OH, alkyl, optionally substituted heteroaryl, phenyl, and haloalkyl, and R₁₆ is H or alkyl.

14. A compound according to claim 13, wherein R₆ is selected from H, F, Cl, CN, methoxy, CH₃, NR₁₁R₁₁′, and CF₃, wherein R₁₁ and R₁₁′ are each independently selected from H and C₁-C₆ alkyl, preferably where R₆ is H.

15. A compound according to claim 13 or 14 wherein R₇ is selected from H, F, Cl, CN, methoxy, CH₃, NR₁₁R₁₁′ and CF₃, preferably where R₇ is H.

16. A compound according to any of claims 13 to 15, wherein R₈ is selected from H, F, OH, CN, methoxy, NR₁₁R₁₁′, phenyl, CF₃, CF₂H, NHSO₂CH₃, NHCOCH₃, and NHCHF₂, wherein R₁₁ and R₁₁′ are each independently selected from H and C₁-C₆ alkyl.

17. A compound according to any of claims 13 to 16, wherein R₉ is selected from H, F, Cl, CN, methoxy, CH₃, NR₁₁R₁₁′ and CF₃, wherein R₁₁ and R₁₁′ are each independently selected from H and C₁-C₆ alkyl, preferably where R₉ is H.

18. A compound according to any one of claims 13 to 17, wherein ring A is selected from groups (vii), (xi) and (xxxiv).

19. A compound according to claim 13, wherein ring A is:

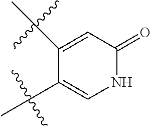 [SMILES: *c1c[nH]c(=O)cc1*]

20. A compound according to claim 13, wherein ring A is:

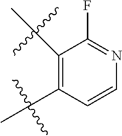 [SMILES: *c1ccnc(F)c1*]

21. A compound according to claim 13, wherein ring A is:

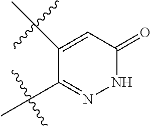 [SMILES: *c1cc(=O)[nH]nc1*]

22. A compound according to any preceding claim wherein Y and Z are each independently selected from CH₂, CHMe, CF₂, C(CH₃)₂, C(CF₃)₂, and are preferably both CH₂.

23. A compound according to any preceding claim, which is selected from the following: 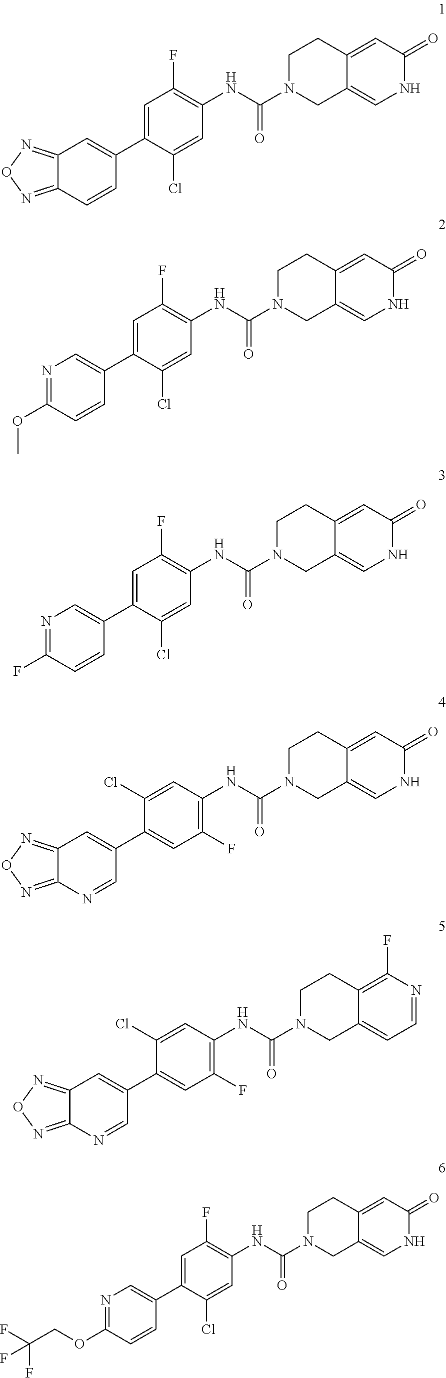 [SMILES: O=C(Nc1cc(Cl)c(-c2ccc3nonc3c2)cc1F)N1CCc2cc(=O)[nH]cc2C1 ; COc1ccc(-c2cc(F)c(NC(=O)N3CCc4cc(=O)[nH]cc4C3)cc2Cl)cn1 ; O=C(Nc1cc(Cl)c(-c2ccc(F)nc2)cc1F)N1CCc2cc(=O)[nH]cc2C1 ; O=C(Nc1cc(Cl)c(-c2cnc3nonc3c2)cc1F)N1CCc2cc(=O)[nH]cc2C1 ; O=C(Nc1cc(Cl)c(-c2cnc3nonc3c2)cc1F)N1CCc2c(ccnc2F)C1 ; O=C(Nc1cc(Cl)c(-c2ccc(OCC(F)(F)F)nc2)cc1F)N1CCc2cc(=O)[nH]cc2C1] 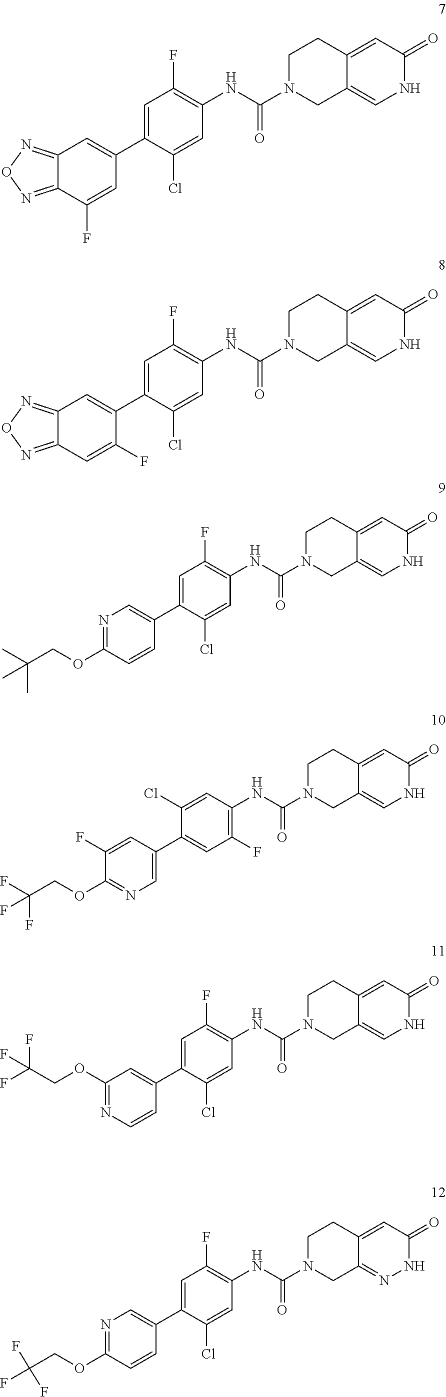 [SMILES: O=C(Nc1cc(Cl)c(-c2cc(F)c3nonc3c2)cc1F)N1CCc2cc(=O)[nH]cc2C1 ; O=C(Nc1cc(Cl)c(-c2cc3nonc3cc2F)cc1F)N1CCc2cc(=O)[nH]cc2C1 ; CC(C)(C)COc1ccc(-c2cc(F)c(NC(=O)N3CCc4cc(=O)[nH]cc4C3)cc2Cl)cn1 ; O=C(Nc1cc(Cl)c(-c2cnc(OCC(F)(F)F)c(F)c2)cc1F)N1CCc2cc(=O)[nH]cc2C1 ; O=C(Nc1cc(Cl)c(-c2ccnc(OCC(F)(F)F)c2)cc1F)N1CCc2cc(=O)[nH]cc2C1 ; O=C(Nc1cc(Cl)c(-c2ccc(OCC(F)(F)F)nc2)cc1F)N1CCc2cc(=O)[nH]nc2C1] 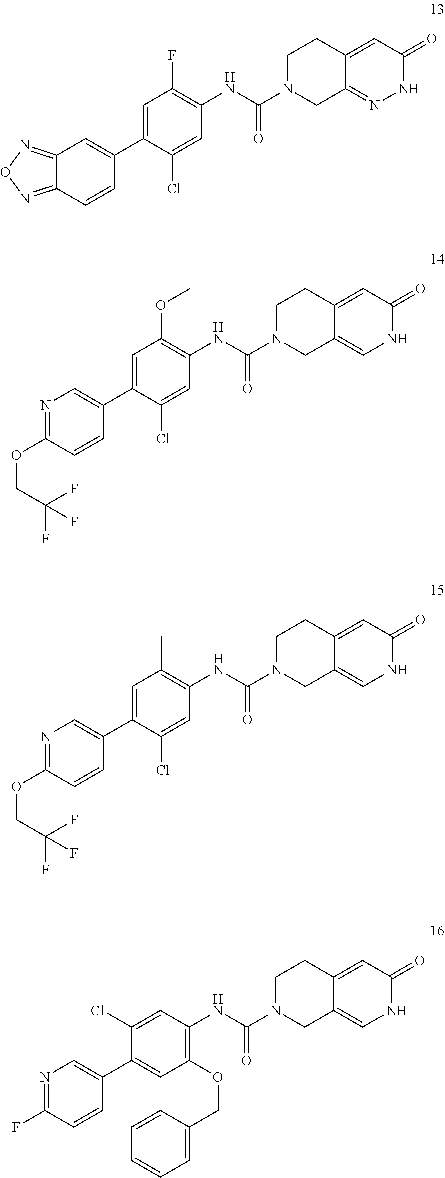 [SMILES: O=C(Nc1cc(Cl)c(-c2ccc3nonc3c2)cc1F)N1CCc2cc(=O)[nH]nc2C1 ; COc1cc(-c2ccc(OCC(F)(F)F)nc2)c(Cl)cc1NC(=O)N1CCc2cc(=O)[nH]cc2C1 ; Cc1cc(-c2ccc(OCC(F)(F)F)nc2)c(Cl)cc1NC(=O)N1CCc2cc(=O)[nH]cc2C1 ; O=C(Nc1cc(Cl)c(-c2ccc(F)nc2)cc1OCc1ccccc1)N1CCc2cc(=O)[nH]cc2C1]
and pharmaceutically acceptable salts and solvates thereof.

24. A compound according to any one of claims 1-23 for use as a medicament.

25. A pharmaceutical composition comprising a compound, or a pharmaceutically acceptable salt or solvate thereof, as defined according to any one of claims 1-23, and a pharmaceutically acceptable diluent, excipient, or carrier.

26. A compound according to any one of claims 1 to 23, or a pharmaceutically acceptable salt or solvate thereof, or a pharmaceutical composition according to claim 25, for use in treating or preventing a disorder selected from a proliferative disorder, an immune disorder, asthma, chronic obstructive pulmonary disease (COPD) and acute respiratory distress syndrome (ARDS).

27. A compound or pharmaceutical composition for use according to claim 26, wherein the compound modulates GPR65, preferably wherein the compound inhibits GPR65 signalling.

28. A compound or pharmaceutical composition for use according to claim 26 or claim 27, wherein the disorder is a proliferative disorder.

29. A compound or pharmaceutical composition for use according to claim 28, wherein the proliferative disorder is a cancer, and is preferably a solid tumour and/or metastases thereof.

30. A compound or pharmaceutical composition for use according to claim 29, wherein the proliferative disorder is a cancer selected from melanoma, renal cell carcinoma (RCC), gastric cancer, acute myeloid leukaemia (AML), pancreatic adenocarcinoma, triple negative breast cancer (TNBC), colorectal cancer, head and neck cancer, colorectal adenocarcinoma, lung cancer, sarcoma, ovarian cancer, and glioma, preferably glioblastoma (GBM).

31. A compound or pharmaceutical composition for use according to claim 26 or claim 27, wherein the disorder is an immune disorder.

32. A compound or pharmaceutical composition for use according to claim 31, wherein the immune disorder is an autoimmune disease.

33. A compound or pharmaceutical composition for use according to claim 32, wherein the autoimmune disease is selected from psoriasis, psoriatic arthritis, rheumatoid arthritis (RA), multiple sclerosis (MS), systemic lupus erythematosus (SLE), autoimmune thyroiditis (Hashimoto's thyroiditis), Graves' disease, uveitis (including intermediate uveitis), ulcerative colitis, Crohn's disease, autoimmune uveoretinitis, systemic vasculitis, polymyositis-dermatomyositis, systemic sclerosis (scleroderma), Sjogren's Syndrome, ankylosing spondylitis and related spondyloarthropathies, sarcoidosis, autoimmune hemolytic anemia, immunological platelet disorders, and autoimmune polyendocrinopathies.

34. A compound or pharmaceutical composition for use according to claim 32, wherein the autoimmune disease is selected from psoriasis, psoriatic arthritis, ankylosing spondylitis, Crohn's disease, and multiple sclerosis.

35. A compound or pharmaceutical composition for use according to claim 26 or claim 27 wherein the use comprises treating or preventing a disorder selected from asthma, chronic obstructive pulmonary disease (COPD) and acute respiratory distress syndrome (ARDS).

36. A method of treating a disorder as defined in any of claims 26 to 35, comprising administering to a subject a compound as defined in any of claims 1-23, or a pharmaceutically acceptable salt or solvate thereof, or a pharmaceutical composition as defined in claim 25.

37. A compound of formula (I) as defined in any one of claims 1-23, or a pharmaceutically acceptable salt or solvate thereof, for use in treating or preventing a GPR65-associated disease or disorder.

38. Use of a compound of formula (I) as defined in any one of claims 1 to 23, or a pharmaceutically acceptable salt or solvate thereof, in the preparation of a medicament for treating or preventing a GPR65-associated disease or disorder in a subject.

39. Use of a compound of formula (I) as defined in any one of claims 1 to 23, or a pharmaceutically acceptable salt or solvate thereof, in the preparation of a medicament for treating or preventing a disorder selected from a proliferative disorder, an immune disorder, asthma, chronic obstructive pulmonary disease (COPD) and acute respiratory distress syndrome (ARDS).
